(19) United States
(12) Patent Application Publication
(10) Pub. No.: US 2025/0041217 A1
(43) Pub. Date: Feb. 6, 2025

(54) Compositions

(71) Applicant: Vitux Group AS, Oslo (NO)

(72) Inventors: Tuna BAYDIN, Oslo (NO); Andrzej SIWEK, Oslo (NO); Magnus HATTREM, Oslo (NO); Kurt Ingar DRAGET, Oslo (NO)

(21) Appl. No.: 18/792,864

(22) Filed: Aug. 2, 2024

Related U.S. Application Data
(60) Provisional application No. 63/530,722, filed on Aug. 4, 2023.

(51) Int. Cl. A61K 9/107; A61K 9/00; A61K 9/06; A61K 47/24; A61K 47/36; A61K 47/44

(52) U.S. Cl. CPC A61K 9/107; A61K 9/0053; A61K 9/06; A61K 47/24; A61K 47/36; A61K 47/44

(57) ABSTRACT

The invention relates to compositions for oral administration which are provided in the form of gelled oil-in-water emulsions, to methods for their preparation and to their use as pharmaceuticals and nutraceuticals. In particular, it relates to an orally administrable, gelled oil-in-water emulsion in unit dose form, wherein the gelled oil-in-water emulsion is a self-supporting, viscoelastic solid having a water activity in the range of about 0.4 to 0.9 and which comprises a gelled aqueous phase having a pH of 3 to 5.5, and wherein the gelled aqueous phase comprises a gelling agent which is a low-methoxy amidated pectin and the gelled oil-in-water emulsion is stabilised by an emulsifier which is a phospholipid or mixture of phospholipids.

### CROSS-REFERENCE TO RELATED APPLICATIONS

This application claims the benefit of priority to U.S. Provisional Application No. 63/530,722 filed Aug. 4, 2023, which is hereby incorporated herein by reference in its entirety.

### FIELD OF THE INVENTION

The present invention generally relates to compositions for oral administration which are provided in the form of gelled oil-in-water emulsions, to methods for their preparation and to their use as pharmaceuticals and nutraceuticals. The compositions are soft, yet chewable, and are provided in a unit dosage form which is easy to swallow.

More specifically, the invention relates to oral compositions which are acceptable to patients and consumers that wish to abstain from the consumption of animal by-products, for example those that follow a vegetarian diet or who are vegan. It also relates to oral compositions that are acceptable to pescetarians.

### BACKGROUND OF THE INVENTION

Soft chewable dosage forms are an alternative to traditional oral administration forms such as tablets, capsules, elixirs and suspensions. They are easier to swallow than tablets and capsules and are particularly suitable for the pediatric and elderly population as well as those that suffer from dysphagia. Such dosage forms are a popular choice for dietary supplements which contain vitamins and/or minerals (so-called “nutraceuticals”), and are also suitable for the delivery of active pharmaceutical ingredients (APIs). Active components (nutraceutical or pharmaceutical) may be present in the form of lipids in gelled oil-in-water emulsions, as dispersed particulates or dissolved in the oil or aqueous phase of such emulsions.

Chewable compositions in unit dose form comprising an oil-in-water emulsion in which the aqueous phase is gelled and in which the oil phase comprises a physiologically tolerable unsaturated fatty acid ester are disclosed in WO 2007/085840, for example. These compositions are suitable for the oral delivery of fish oils without any accompanying bad odor or taste. In WO 2010/041015 and WO 2012/140392, drug substances are dissolved or dispersed in the oil or aqueous phase of gelled oil-in-water emulsions in order to taste mask the drug substances.

A range of gelling agents can be used in the preparation of soft chewable dosage forms, such as the gelled oil-in-water emulsions described in WO 2007/085840, WO 2010/041015 and WO 2012/140392, however gelatin is by far the most widely used due to its availability, ease of use and its sol-gel transition temperature.

Gelatin is produced by partial hydrolysis of collagen found in the skin, bones and connective tissue of animals and is most commonly derived from pork, bovine and fish sources. The sol-gel transition temperature of a gelatin generally corresponds to the body temperature of the animal from which it is obtained. Gelatins from mammalian sources therefore have transition temperatures which are similar to human body temperature, resulting in gels which are solid at room temperature but which melt in the mouth once ingested. Gelatin-based dosage forms thus provide a pleasant ‘melt-in-the mouth’ texture or “mouthfeel”.

Despite the advantageous properties of gelatin for use in the production of soft chewable dosage forms, its use is associated with certain drawbacks. For example, its low sol-gel transition temperature can impact its use in products that are produced or consumed in warm and hot climate countries. If the temperature reaches above 40° C., it may become necessary to refrigerate the product. This is inconvenient and costly both in terms of transport and storage. The animal origin of gelatin also makes it unacceptable to many patients and consumers due to their religious beliefs or dietary choices. As an animal by-product, gelatin is not acceptable to vegans for example. Gelling agents that are not of animal origin are thus desirable for use in the production of soft chewable dosage forms, such as gelled oil-in-water emulsions.

The type of gelled oil-in-water emulsions disclosed in WO 2007/085840, WO 2010/041015 and WO 2012/140392 represent a particular challenge when seeking to replace the gelatin with other gelling agents. In order to reduce the potential for microbial growth and thus extend shelf-life, these gelled oil-in-water emulsions have a low water activity (i.e. a low content of ‘free’ water). This is achieved by the inclusion of a high content of sugar alcohols (bulking agents) in the gelled aqueous phase. As a result, these gelled emulsions differ from most conventional oil-in-water emulsions in that they have a low water activity (a_{w} below 0.8, for example, typically about 0.75). The presence of buffer salts (pH adjusting agents) in the aqueous phase also results in a relatively low pH (typically between 4 and 4.7). Together, these factors make it difficult to find alternative combinations of food or pharmaceutical grade gelling agents and emulsifiers that are capable of providing a stable emulsion under these conditions.

In the gelatin-based emulsions disclosed in WO 2007/085840, WO 2010/041015 and WO 2012/140392, the pH of the aqueous phase must be kept within a narrow range (typically from about 4.5 to 4.8) in order to avoid degradation of the gelatin. Some components, such as certain nutraceutical agents, are not stable in this pH range and require a lower pH for stability. This can limit the type of agents which may be included in known gelatin-based emulsions.

A continuing need thus exists for alternative soft, yet chewable, compositions for the oral delivery of pharmaceuticals and/or nutraceuticals that are suitable for vegetarians, pescetarians and vegans. In particular, there is a need for such compositions that can provide an acceptable alternative to conventional gelatin-based oil-in-water emulsions in terms of their “chew” and mouthfeel characteristics. Such compositions should be capable of manufacture on a commercial scale and have adequate stability (i.e. shelf-life) for use as pharmaceutical and/or nutraceutical products. In particular, there is a need for such compositions that have better thermal stability than gelatin-based compositions and which allow for the administration of certain ingredients that require a lower pH for stability.

The present invention addresses at least some of these needs.

### SUMMARY OF THE INVENTION

The Applicant now proposes gelled oil-in-water emulsions that are acceptable to patients and consumers that are vegetarian, pescetarian or vegan, in particular to those that follow a vegetarian or vegan diet. As a gelling agent, the emulsions employ pectin having a low degree of esterification and which is partially amidated. Specifically, the emulsions contain a low-methoxy amidated pectin as the gelling agent and are stabilised using an emulsifier which is a phospholipid or mixture of phospholipids. The Applicant has found that this gelling agent/emulsifier combination is particularly effective in providing a stable, gelled oil-in-water emulsion having low water activity and low pH, and which has desirable rheology characteristics for oral delivery.

In one aspect the invention provides an orally administrable, gelled oil-in-water emulsion in unit dose form, wherein the gelled oil-in-water emulsion is a self-supporting, viscoelastic solid having a water activity in the range of about 0.4 to 0.9 and which comprises a gelled aqueous phase having a pH of 3 to 5.5, and wherein the gelled aqueous phase comprises a gelling agent which is a low-methoxy amidated pectin and the gelled oil-in-water emulsion is stabilised by an emulsifier which is a phospholipid or mixture of phospholipids.

In another aspect the invention provides a method for the preparation of a gelled oil-in-water emulsion as herein described, said method comprising the steps of: forming an oil phase which comprises one or more physiologically tolerable lipids; forming an aqueous phase comprising a gelling agent which is a low-methoxy amidated pectin; combining said oil phase and said aqueous phase in the presence of an emulsifier which is a phospholipid or mixture of phospholipids to form an oil-in-water emulsion; and allowing said emulsion to gel.

In a further aspect the invention provides a gelled oil-in-water emulsion as herein described for oral use as a medicament or for oral use in therapy.

In another aspect the invention provides a gelled oil-in-water emulsion as herein described which contains at least one pharmaceutically active component for oral use in the treatment of a condition responsive to said pharmaceutically active component.

In another aspect the invention provides the use of a pharmaceutically active component in the manufacture of a medicament for oral use in the treatment of a condition responsive to said pharmaceutically active component, wherein said medicament is provided in the form of a gelled oil-in-water emulsion as herein described.

In another aspect the invention provides a method of treatment of a human or non-human animal subject (e.g. a patient) to combat a condition responsive to a pharmaceutically active agent, said method comprising the step of orally administering to said subject a pharmaceutically effective amount of said agent in the form of a gelled oil-in-water emulsion as herein described.

In another aspect the invention provides the use of a gelled oil-in-water emulsion as herein described as a nutraceutical.

### BRIEF DESCRIPTION OF THE FIGURES

The accompanying figures, which are incorporated in and constitute a part of this specification, illustrate several aspects of the disclosure, and together with the description, serve to explain the principles of the disclosure.

FIG. 1A. Large scale deformation of the gels with texture analyzer. 75% strain single compression was applied. The curves are averages of 6-10 replicates (obtained from a single batch). The abbreviations and compositions of the gels are given in Table 8.

FIG. 1B. Texture profile analysis of the gels with 20% strain. Each curve represents the average of 7-10 gels (obtained from a single batch). The abbreviations and compositions of the gels are given in Table 8.

FIG. 2A. Total NaOH consumption of agar (AEG), pectin (PEG) and gelatin (GEG) emulsion gels during in vitro lipolysis in gastrointestinal stage. The curves show total NaOH consumption without subtracting the consumption of the corresponding control gels.

FIG. 2B. Total NaOH consumption of agar (AEG), pectin (PEG) and gelatin (GEG) emulsion gels during in vitro lipolysis in intestinal stage. The curves show total NaOH consumption without subtracting the consumption of the corresponding control gels.

FIG. 3A. Photomicrograph of agar emulsion gel after the gastric stage. 20× objective was used.

FIG. 3B. Photomicrograph of pectin emulsion gel after the gastric stage. 20× objective was used.

FIG. 3C. Photomicrograph of gelatin emulsion gel after the gastric stage. 20× objective was used.

FIG. 4. Strain sweep of 7.15% agar gel (AG).

FIG. 5. Texture profile analysis of the gels with 30% strain. Each curve represents the average of 7-10 gels (obtained from a single batch). The abbreviations and compositions of the gels are given in Table 8.

FIG. 6. Texture profile analysis of the gels with 75% strain. Each curve represents the average of 7-10 gels (obtained from a single batch). The abbreviations and compositions of the gels are given in Table 8.

FIG. 7. Force after gel deformation at strains from 30 to 70%. The maximum force at single compression was regarded as 1 and force after deformation was normalized to 1. The abbreviations and compositions of the gels are given in Table 8.

### DETAILED DESCRIPTION OF THE INVENTION

### Definitions

The term “gel” refers to a form of matter that is intermediate between a solid and a liquid. The formation of a “gel” will typically involve the association or cross-linking of polymer chains to form a three-dimensional network that traps or immobilises solvent (e.g. water) within it to form a sufficiently rigid structure that is resistant to flow at ambient temperature, i.e. at a temperature below about 25° C., preferably below about 20° C. In rheological terms, a “gel” may be defined according to its storage modulus (or “elastic modulus”), G′, which represents the elastic nature (energy storage) of a material, and its loss modulus (or “viscous modulus”), G″, which represents the viscous nature (energy loss) of a material. Their ratio, tan δ (equal to G″/G′), also referred to as the “loss tangent”, provides a measure of how much the stress and strain are out of phase with one another.

A material which is “viscoelastic” is characterised by rheological properties which resemble, in part, the rheological behaviour of a viscous fluid and, also in part, that of an elastic solid.

The gelled oil-in-water emulsions according to the invention are “self-supporting, viscoelastic solids”. This is intended to mean that they exhibit characteristics intermediate between those of a solid and a liquid, but have a dominant solid behaviour, i.e. they have rheological characteristics more similar to that of a solid than a liquid. A “solid dominant behaviour” cannot be diluted away (i.e. destroyed) by adding more solvent. In contrast, in the case of a weak (or entangled) gel lacking stable (i.e. long lived) intermolecular crosslinks, the entangled network structure of the gel can be removed by adding more solvent and can readily be destroyed even at very low shear rate/shear stress.

The gelled oil-in-water emulsions of the invention exhibit mechanical rigidity, yet in contrast to a solid they are deformable. Specifically, the gelled emulsions herein described have a storage modulus, G′, which is greater than their loss modulus, G″, (i.e. G′>G″) over a wide frequency range, for example in the frequency range from 0.001 to 10 Hz when measured at ambient temperature (i.e. at a temperature in the range of 18° C. to 25° C., e.g. at 20° C.) and 0.1% strain. Storage modulus and loss modulus may be measured using known methods, for example using a Kinexus Ultra+ Rheometer applying a C 4/40 measuring geometry. Storage modulus and loss modulus values are not expected to differ when measured using other types of rheometer within the linear viscoelastic range.

More specifically, the gelled oil-in-water emulsions herein described will have the following properties: G′>G″ over a frequency range of 0.001 to 10 Hz at 0.1% strain; and a storage modulus (G′) at ambient temperature (i.e. at a temperature in the range of 18° C. to 25° C., e.g. at 20° C.) in the range from 10 to 200,000 Pa, preferably 100 to 100,000 Pa, more preferably 500 to 50,000 Pa.

Weak gels will typically have a loss tangent, tan δ>0.1. For strong gels, or fully developed gels, G′>>G″ and lower tan δ values (<0.1) are observed. The gelled oil-in-water emulsions herein described would generally be considered “strong gels” at ambient temperature, i.e. at a temperature in the range of 18° C. to 25° C., e.g. at 20° C.

As used herein, the term “gelled” refers to the formation of a “gel”. The term is used herein both in relation to the physical nature of the aqueous phase of the emulsion and that of the oil-in-water emulsion. As will be understood, the oil droplets act more or less like a solid when dispersed throughout the gelled aqueous phase of the oil-in-water emulsions which are the subject of the invention. The “gelled” nature of the aqueous phase is thus also a characteristic of the oil-in-water emulsion, i.e. it can also be considered “gelled” as described herein.

Unless otherwise defined, the term “liquid” as used herein refers to a substance which flows freely and which maintains a constant volume. It includes thickened liquids and viscous liquids which flow. A “liquid” has a loss modulus (G″) which is greater than its storage modulus (G′) and a loss tangent (tan δ) which is greater than 1.

As used herein, the term “water activity” (“a_{w}”) refers to the partial vapour pressure of water in a composition at a specified temperature divided by the standard state partial vapour pressure of water at the same temperature. Water activity thus acts as a measure of the amount of free (i.e. unbound) water in a composition. It may be calculated as:

a_{w}=p/p₀
where p is the partial vapour pressure of water in the composition and p₀ is the vapour pressure of pure water at the same temperature.

Alternatively, water activity may be calculated as:

a_{w}=I_{w}x_{w}
where I_{w} is the activity coefficient of water and x_{w} is the mole fraction of water.

Water activity may be measured by methods known to those skilled in the art, for example by using a Rotronic Hygrolab instrument. Unless otherwise specified, water activity is measured at ambient temperature, i.e. 21° C.±2° C.

As used herein, the term “pH” is a measure of the concentration of hydrogen ions in an aqueous medium at ambient temperature, i.e. 21° C.±2° C. pH may be measured by methods known to those skilled in the art, for example using a contact electrode for pH measurement such as a calibrated Eutech Instruments pHSpear.

As used herein, the term “emulsifier” refers to a surface-active compound or composition which is capable of reducing the interfacial tension between two immiscible liquids, e.g. at the interface between oil and water. Emulsifiers aid both the droplet break-up of the discontinuous phase as well as stabilising the resulting emulsion. An emulsifier may consist of a single component or may be a mixture of components. Where the emulsifier is a mixture of components, the individual components will typically, though not necessarily, be similar in structure. When the emulsifier for use in the invention is obtained from a natural product (for example, from a plant or plant part), it will be understood that it will typically comprise a mixture of different components. The emulsifier may, for example, be a naturally-occurring product obtained from a plant or part of a plant, or it may be a derivative thereof as described herein (i.e. it may be semi-synthetic).

As used herein, the term “fatty acid” refers to an un-branched or branched, preferably un-branched, hydrocarbon chain having a carboxylic acid (—COOH) group at one end, conventionally denoted the a (alpha) end. The hydrocarbon chain may be saturated or (mono- or poly-) unsaturated. By convention, the numbering of the carbon atoms starts from the α-end such that the carbon atom of the carboxylic acid group is carbon atom number 1. The other end, which is usually a methyl (—CH₃) group, is conventionally denoted ω (omega) such that the terminal carbon atom is the ω-carbon. Any double bonds present may be cis- or trans-in configuration. The nomenclature “ω-x” is used to signify that a double bond is located on the xth carbon-carbon bond, counting from the terminal carbon (i.e. the ω-carbon) towards the carbonyl carbon.

By “physiologically tolerable” is meant any component which is suitable for administration to a human or non-human animal body, in particular which is suitable for oral administration.

By “pharmaceutical” is meant any product intended for a medical purpose, e.g. for treating or preventing any disease, condition or disorder of a human or non-human animal body, or for preventing its recurrence, or for reducing or eliminating the symptoms of any such disease, condition or disorder. The use and production of a product as a “pharmaceutical” will be closely regulated by a government agency. It may, but need not, be prescribed by a physician. For example, it may be available “over the counter”, i.e. without a prescription.

“Treatment” or “treating” includes any therapeutic application that can benefit a human or non-human animal (e.g. a non-human mammal). Both human and veterinary treatments are within the scope of the present invention, although primarily the invention is aimed at the treatment of humans. Veterinary treatment includes the treatment of livestock and domestic animals (e.g. pets such as cats, dogs, rabbits, etc.). Treatment may be in respect of an existing disorder or it may be prophylactic.

In contrast to a pharmaceutical, a “nutraceutical” need not be the subject of regulatory approval. The term “nutraceutical” is used herein to refer to a product which is generally considered beneficial to maintain or augment the health and/or general well-being of a human or non-human animal subject. Such substances include, in particular, dietary supplements such as vitamins and minerals which are intended to augment the health of a subject (e.g. a human subject).

As will be understood, some substances may be considered both a “pharmaceutical” and a “nutraceutical”. Categorization of a substance as one or the other, or indeed both, may vary in different countries depending on local regulations relating to medicinal products. It may also be dependent on the recommended daily dosage of any given substance. Higher daily doses of certain vitamins such as vitamin D, for example, may be regulated as a pharmaceutical whereas lower daily dosages may be considered nutraceutical.

By “a pharmaceutical composition” is meant a composition in any form suitable to be used for a pharmaceutical purpose.

By a “nutraceutical composition” is meant a composition in any form suitable to be used for a nutraceutical purpose.

A “pharmaceutically effective amount” relates to an amount that will lead to the desired pharmacological and/or therapeutic effect, i.e. an amount of the agent which is effective to achieve its intended pharmaceutical purpose. While individual patient needs may vary, determination of optimal ranges for effective amounts of any active agent is within the capability of those skilled in the art.

A “nutraceutically effective amount” relates to an amount that will lead to the desired nutraceutical effect, i.e. an amount of the agent which is effective to achieve its intended nutraceutical purpose. While the individual needs of a subject may vary, determination of optimal ranges for effective amounts of any active agent is within the capability of those skilled in the art.

The term “capsule” is used herein to refer to a unitary dosage form having a casing or coating (herein referred to as the “capsule shell”) which encloses a gelled oil-in-water emulsion as herein defined.

As used herein, an “animal by-product” is intended to refer to any product derived from, isolated from, or purified from one or more parts of an animal body (e.g. bone, skin, tissue, meat, cartilage, hoof, horn, etc.). It is also intended to refer to any composition prepared by processing an animal by-product, for example derivatised, functionalised, or otherwise chemically or physically modified, animal by-products. As used herein, an “animal by-product” is not intended to include milk, eggs, or any compound or composition that is derived from, isolated from, or purified from animal milk or animal eggs. The term “animal by-product” does not include any synthetic material, or any material obtained from any plant, fungal, bacterial or algal source.

As used herein, the term “vegetarian diet” generally refers to a diet that lacks any meat and which also lacks any animal by-product as herein defined. A “vegetarian diet” may include animal milk and animal eggs and any products derived, isolated or purified therefrom. Such a diet may also be generally known as an “ovo-lactovegetarian” diet or “lacto-ovovegetarian” diet which, in addition to food from plants, includes milk, cheese, other dairy products and eggs. A “pescetarian diet” refers to a diet in which the only source of meat is fish and seafood. A “vegan diet” refers to a diet that is totally vegetarian and which includes only food from plants (e.g. fruit, vegetables, grains, legumes, seeds and nuts). Any reference herein to a product, substance, composition or formulation which is “suitable for” a given diet means that it would be acceptable for those that follow that particular diet.

The terms “vegetarian”, “pescetarian” and “vegan” are intended to refer to those who follow a vegetarian, pescetarian or vegan diet, respectively.

In a first aspect the invention provides an orally administrable, gelled oil-in-water emulsion in unit dose form, wherein the gelled oil-in-water emulsion is a self-supporting, viscoelastic solid having a water activity in the range of about 0.4 to 0.9 and which comprises a gelled aqueous phase having a pH of 3 to 5.5, and wherein the gelled aqueous phase comprises a gelling agent which is a low-methoxy amidated pectin and the gelled oil-in-water emulsion is stabilised by an emulsifier which is a phospholipid or mixture of phospholipids.

The aqueous phase of the emulsion according to the invention comprises water and is gelled using low-methoxy amidated pectin as a gelling agent. The aqueous phase is also referred to herein as the “continuous phase” of the emulsion. Typically, the low-methoxy amidated pectin will be the only type of gelling agent used in the emulsion, i.e. it will be the sole gelling agent.

Pectin is well known and used in the art, for example in food and other non-food applications. As used herein, the term “pectin” is intended to broadly define a group of heteropolysaccharides found in plants and having a backbone composed predominantly of polygalacturonic acid (for example, 60-70% galacturonic acid). Besides galacturonic acid residues, pectins may contain L-rhamnose residues which form part of the backbone of the polysaccharide. D-galacturonic and L-rhamnose residues may act as sites for branching comprising other sugars such as D-glucose, D-galactose, L-arabinose, D-xylose, D-mannose, and L-fucose. The occurrence of such residues varies depending on the source of the pectin.

In nature, about 80% of the carboxyl groups of galacturonic acid are esterified with methanol but this proportion is decreased to a varying degree during pectin extraction. Pectins are classified as high or low methoxy pectin (“HM-pectin” and “LM-pectin”, respectively) according to their degree of esterification. The ratio of methyl esterified galacturonic acid groups to the total galacturonic acid groups is the degree of esterification. As used herein, a “low-methoxy” pectin refers to pectin having a degree of esterification of less than 50%. A “high-methoxy” pectin, as referred to herein, is one having a degree of esterification of at least 50%.

High methoxy pectin forms a gel under acidic conditions (i.e. at a low pH), whereas low methoxy pectin mainly forms gels by interaction with divalent cations such as calcium ions in which ionic linkages are formed between calcium ions and carboxyl groups of the galacturonic acid residues. When the pectin is amidated, gel formation is also induced by hydrogen bonding between amide groups. Its gelling is therefore less dependent on calcium concentration.

The Applicant has found that a low-methoxy amidated pectin is particularly suitable for use in the production of the gelled oil-in-water emulsions herein described. In particular, the “heat reversible” gelling of this type of pectin aids in the production process. As used herein, the term “heat reversible” refers to a gelling agent that is liquid at elevated temperature but forms a gel on cooling and which melts again when re-heated. In contrast, the gelling of high methoxy pectin, which is dependent on a lowering of the pH, is non-reversible.

Pectin is found in many plants, and particularly in fruits such as apples, lemons and limes in which it is located in the primary cell walls in the form of water-insoluble protopectin. Water soluble pectin is obtained by the acid or alkaline hydrolysis of protopectin. Depending on the processing conditions (pH, temperature and time of extraction, etc.), pectin having different degrees of esterification is obtained.

Sources of the raw material from which pectin may be obtained include the peel, pulp and/or rag from citrus fruits such as lemons, oranges, mandarins, limes, grapefruits, tangerines, apple and pear pomace, potato peel, sugar beet pulp, grape skin, pea pods, and carrot fibre. Whole fruits and vegetables such as tomatoes, quinces, mangoes, pineapples, plums and strawberries are also suitable as raw material sources. In one embodiment, the source of the pectin for use in the invention is citrus fruit peel. This has a high content of water-insoluble protopectin.

The pectin for use in the invention is low-methoxy amidated pectin. As herein defined, this means that the pectin has a degree of esterification of less than 50%, i.e. less than 50% of the galacturonic acid residues are present in the form of a methyl ester. The non-esterified galacturonic acid units may be present in the form of free carboxyl groups or as salts with mono or divalent cations such as sodium, potassium or calcium ions.

In one set of embodiments, the low-methoxy amidated pectin for use in the invention has a degree of esterification of less than 40%. In one set of embodiments, the pectin for use in the invention has a degree of esterification of less than 35%. In one set of embodiments, the pectin for use in the invention has a degree of esterification of less than 30%. In one set of embodiments, the pectin for use in the invention has a degree of esterification in the range of from 10% to 40%. In one set of embodiments, the pectin for use in the invention has a degree of esterification in the range of from 15% to 35%. In one set of embodiments, the pectin for use in the invention has a degree of esterification in the range of from 20% to 30%. In one set of embodiments, the pectin for use in the invention has a degree of esterification of about 20%, about 21%, about 22%, about 23%, about 24%, about 25%, about 26%, about 27%, about 28%, about 29% or about 30%.

As used herein, the term “amidated pectin” refers to a pectin in which at least a proportion of the methyl ester groups have been converted to amide groups. The degree of amidation is defined as the ratio of amidated galacturonic acid groups to the total galacturonic acid groups.

In one set of embodiments, the pectin for use in the invention has a degree of amidation in the range of 15 to 25%. In one set of embodiments, the pectin for use in the invention has a degree of amidation in the range of 20 to 25%. In one set of embodiments, the pectin for use in the invention has a degree of amidation of about 20%, about 21%, about 22%, about 23%, about 24%, or about 25%.

In one set of embodiments, the pectin for use in the invention has a degree of esterification in the range of from 10% to 40% and a degree of amidation in the range of from 15 to 25%. In one set of embodiments, the pectin for use in the invention has a degree of esterification in the range of from 15% to 35% and a degree of amidation in the range of range of from 20 to 25%. In one set of embodiments, the pectin for use in the invention has a degree of esterification in the range of from 20% to 30% and a degree of amidation in the range of from 20 to 25%. In one set of embodiments, the pectin for use in the invention has a degree of esterification of about 20%, about 21%, about 22%, about 23%, about 24%, about 25%, about 26%, about 27%, about 28%, about 29% or about 30%, and a degree of amidation of about 20%, about 21%, about 22%, about 23%, about 24% or about 25%.

Commercial pectins are typically standardised by the addition of sugars such as sucrose. This provides pectins having a standard gel strength. Unless otherwise indicated, any reference herein to the concentration of pectin refers to the amount of pectin in the composition, i.e. not the amount of ‘standardised’ pectin.

Pectin for use in the invention can be obtained from various commercial sources. A non-limiting example of a pectin which may be used is GENU® pectin type LM-104 AS-FS (CP Kelco ApS or CP Kelco US). This is a partially amidated low ester pectin extracted from citrus peel and standardised by the addition of sucrose. Its degree of esterification is typically 26%. Its degree of amidation is typically 22%.

Low-methoxy pectin typically requires the presence of divalent cations such as Ca²⁺ for gelling. Such cations may be considered a “gelling initiator”. In some embodiments, the aqueous phase of the gelled emulsion will thus further comprise a gelling initiator. The source of the calcium ions in the composition may be one or more of the other components of the gelled emulsion, or it may be an added calcium source, or it may originate from a combination of both. Any source of Ca²⁺ ions may be used, for example a water-soluble calcium compound or salt such as tricalcium citrate, calcium stearate, calcium chloride or calcium lactate.

The required concentration of calcium ions in the emulsion will be dependent on the weight of the pectin and can be selected accordingly. Typically, the total amount of available calcium ions will be 1 to 10 wt. %, preferably 1 to 5 wt. % (based on the weight of the pectin). Where any Ca²⁺ ions are present in the aqueous phase these will typically be present at a concentration in the range of up to 250 mM, preferably 10 to 200 mM, e.g. 25 to 150 mM.

In one set of embodiments, no calcium is required in order to provide the gelled oil-in-water emulsion according to the invention.

The pectin gelling agent will be present in the aqueous phase in an amount suitable to provide the desired degree of gelling as herein described. The amount will vary to some extent dependent on the precise nature of the gelling agent (for example, the type of pectin which is employed) and/or other components of the aqueous phase, but a suitable amount may readily be determined by those skilled in the art.

In one set of embodiments, the low-methoxy amidated pectin may be present in the aqueous phase at a concentration of about 1 to about 10 wt. %, preferably about 1 to about 5 wt. % (i.e. based on the weight of the aqueous phase). The concentration of the low-methoxy amidated pectin based on the overall weight of the composition may range from about 0.5 to about 5 wt. %, preferably from about 1.0 to about 3.5 wt. %, more preferably from about 1.5 to about 2.5 wt. %, e.g. from about 1.5 to about 2.0 wt. %. For example, it may be present at a concentration of 1.50, 1.55, 1.60, 1.65, 1.70, 1.75, 1.80, 1.85, 1.90, 1.95 or 2.0 wt. % (based on the overall weight of the composition).

The gelled oil-in-water emulsions herein described are stabilised by an emulsifier (also referred to herein as an “emulsifying agent”). The emulsifying agent for use in the invention contains a phospholipid or mixture of phospholipids. Phospholipids generally consist of a glycerol molecule linked to two fatty acids (the “tail” groups) and to a hydrophilic “head” group which consists of a phosphate group. The phosphate group may be modified by linkage to choline, ethanolamine or serine to produce phosphatidylcholine, phosphatidylethanolamine and phosphatidylserine. Natural phospholipids have a fatty acid composition that is characteristic of the source from which they are extracted.

Phospholipids having a positive charge, for example phosphatidylcholine, are particularly preferred for use in the invention.

In one set of embodiments, the emulsifying agent for use in the invention is lecithin or a lecithin derivative. Lecithin is a complex mixture of acetone-insoluble phospholipids which consist mainly of phosphatidylcholine, phosphatidylethanolamine, phosphatidylinositol, phosphatidylserine and phosphatidic acid. As a result of its extraction it may also contain various amounts of other substances such as triglycerides, fatty acids and carbohydrates. Phosphatidylcholine is the main component of lecithin. Due to the heterogeneous composition of lecithins, they act as a mixture of emulsifiers and co-emulsifiers.

Lecithins are produced by extraction from various sources such as soybean, sunflower seeds and egg yolk. Extraction is typically performed using solvents such as ethanol. The phospholipids may be purified using chromatographic methods in order to obtain specific phospholipid fractions.

Plant lecithins are particularly preferred for use in the invention. In one embodiment, the emulsifier is a lecithin obtained from sunflower seeds, i.e. sunflower lecithin. In one embodiment, the emulsifier is lecithin obtained from soybean, i.e. soy lecithin. In one embodiment, the emulsifier is lecithin obtained from egg, i.e. egg lecithin.

Lecithin derivatives include hydrolysed lecithin. Lecthin may be hydrolysed enzymatically, for example using a phospholipase. In hydrolysed lecithin a portion of the phospholipids have a fatty acid removed.

Lecithin and lecithin derivatives for use in the invention can be obtained from various commercial sources. Non-limiting examples of lecithin derivatives which may be used include: LECICO SUN FM 580 (LECICO GmbH, Germany)—a fluid enzymatically modified multi-functional sunflower lecithin; GIRALEC SRN (Lasenor); and GIRALEC HE-60 (Lasenor).

The emulsifying agent is present in an amount effective to provide the desired stability to the emulsion. The amount will vary dependent on factors such as the precise nature of the emulsifier, the relative proportions of the oil and aqueous phase, and the presence (and amount) of any other components of the emulsion that may act as an emulsifying agent. Taking account of these factors, an appropriate amount of the emulsifying agent may readily be determined by those skilled in the art. A suitable amount may, for example, be in the range from 0.01 to 0.5 wt. %, preferably from 0.05 to 0.4 wt. %, particularly from 0.1 to 0.3 wt. %, e.g. from 0.2 to 0.3 wt. % (based on the total weight of the overall composition). For example, the amount of the emulsifying agent may be 0.1, 0.15, 0.2. 0.25 or 0.3 wt. %, based on the total weight of the composition.

The oil phase of the emulsion will comprise a physiologically tolerable lipid, or a mixture of different physiologically tolerable lipids. Depending on the nature of the lipid (or lipids), the oil phase itself may have nutraceutical and/or pharmaceutical properties. In some embodiments, therefore, the lipids which constitute the oil phase of the emulsion may be the nutraceutical or pharmaceutical agent. Examples of such lipids include, for example, essential fatty acids such as those which are herein described. Alternatively, the oil phase may act as a carrier for a lipophilic pharmaceutical or nutraceutical agent. In this case, the active agent may be dissolved or dispersed in the oil phase.

A range of different lipids are known for oral use in pharmaceutical and/or nutraceutical products and any of these may be used in the oil phase of the emulsions herein described. Sources of lipids include plant oils, such as but not limited to, rapeseed oil, sunflower oil, corn oil, olive oil, sesame oil, palm kernel oil, coconut oil, nut oils (e.g. almond oil or peanut oil), algal oil and hemp oil. Fish oils and lipids obtained from fish oils are also suitable for use in certain compositions according to the invention. Compositions containing these products are acceptable to pescetarians, for example.

Lipids derived from natural products typically comprise a mixture of different lipid components. In one embodiment, the oil phase will thus comprise a mixture of different lipids. For example, it may comprise a mixture of lipids having different chain lengths and/or different degrees of saturation.

Lipids for use in the invention include, in particular, fatty acids and their derivatives. These include both naturally occurring fatty acids and their derivatives, as well as synthetic analogues. In one embodiment, the oil phase may comprise a mixture of different fatty acids, or fatty acid derivatives.

The hydrocarbon chain of the fatty acid or fatty acid derivative may be saturated or unsaturated, but typically it will be unsaturated, and it may be un-branched or branched. Preferably, it will be un-branched. Typically the hydrocarbon chain will comprise from 4 to 28 carbon atoms, and generally it will have an even number of carbons. Fatty acids differ in their chain length and may be categorized as “short”, “medium”, “long”, or “very long” chain fatty acids. Those having a hydrocarbon chain of 5 or fewer carbons are referred to as “short-chain fatty acids”; those with a hydrocarbon chain of 6 to 12 carbon atoms are referred to as “medium-chain fatty acids”; those with a hydrocarbon chain of 13 to 21 carbons are referred to as “long-chain fatty acids”; and those with a hydrocarbon chain of 22 carbons or more are referred to as “very long-chain fatty acids”. Any of these may be used in the invention.

In one embodiment, the oil phase may comprise an unsaturated fatty acid or derivative thereof in which the carbon chain contains one or more carbon-carbon double bonds. The double bonds may be in the cis- or trans-configuration, or any combination thereof where more than one double bond is present. Those in which the double bonds are present in the trans-configuration are generally less preferred due to the need to reduce the consumption of so-called “trans-fats” as part of a healthy diet. Fatty acids and their derivatives having cis-configuration double bonds are thus preferred. Mono- and poly-unsaturated fatty acids and their derivatives are well known in the art. Such fatty acids typically will contain 12 to 26 carbons, more typically 16 to 22 carbons, and will have a mono- or poly-unsaturated hydrocarbon chain. They include, in particular, the polyunsaturated fatty acids (PUFAs) such as the essential fatty acids.

Oils which contain long chain, unsaturated fatty acids and their derivatives find particular use in the invention, for example in any composition which is intended for use as a nutraceutical. Particularly important essential fatty acids which may be used include the ω-3, ω-6 and ω-9 fatty acids. Examples of ω-3 fatty acids include alpha-linolenic acid (ALA), stearidonic acid (SDA), eicosatrienoic acid (ETE), eicosatetraenoic acid (ETA), eicosapentaenoic acid (EPA), docosapentaenoic acid (DPA), docosahexaenoic acid (DHA), tetracosapentaenoic acid and tetracosahexaenoic acid. Examples of ω-6 fatty acids include linoleic acid, gamma-linolenic acid, eicosadienoic acid, dihomo-gamma-linolenic acid (DGLA), arachidonic acid (AA), docosadienoic acid, adrenic acid, docosapentaenoic acid, and calendic acid. Examples of ω-9 fatty acids include oleic acid, eicosenoic acid, mead acid, erucic acid and nervonic acid.

Sources of unsaturated fatty acids and their derivatives include oils obtained from various fish, plant, algae, and microorganism sources. Particularly suitable sources are algae oils and plant oils, however fish oils may also be suitable for those that follow a pescetarian diet. These oils are all rich in ω-3, ω-6 and ω-9 fatty acids. Fish oils may, for example, be obtained from anchovies, sardines and mackerel. Plant oils may be obtained from sunflower seeds, for example.

Any known derivatives of the fatty acids may be used in the invention. These include, in particular, the carboxylic esters, carboxylic anhydrides, and glycerides (i.e. mono-, di-, or triglycerides). As used herein the term “derivatives” in the context of a fatty acid also encompasses any pharmaceutically acceptable salt of a fatty acid. Suitable salts are well known to those skilled in the art and include, but are not limited to, the lithium, sodium, potassium, ammonium, meglumine, and diethylamine salts.

The amount of oil present in the compositions of the invention will be dependent on factors such as the nature of the oil, the nature and desired loading level of any pharmaceutical or nutraceutical that may be present, etc. and can be varied according to need. The oil phase may, for example, constitute from 1 to 40 wt. %, preferably from 5 to 30 wt. %, for example from 10 to 25 wt. %, from 15 to 25 wt. % or from 20 to 25 wt. % of the gelled oil-in-water emulsion.

As will be understood, in the compositions of the invention the oil provides the discontinuous phase within a continuous aqueous phase which is gelled. The oil is thus dispersed throughout the gelled aqueous phase in the form of oil droplets (also referred to herein as oil “particles”). Gelling of the aqueous phase provides a stable emulsion which prevents coalescence of the droplets of oil, for example due to the prevention of physical collisions between droplets.

The size of the oil particles in the gelled oil-in-water emulsion is not particularly limited. For example, oil particles having a volume-based average particle size in the range from about 100 nm to about 100 μm, preferably from about 500 nm to about 50 μm, in particular from about 1 μm to about 25 μm may be provided. “Volume-based average” as used herein refers to the volume moment mean or De Brouckere Mean Diameter (also known as the “D [4,3]” value). This reflects the size of those particles which constitute the bulk of the sample volume and is most sensitive to the presence of large particles in the size distribution. Lipid droplet size and size distributions can be determined using methods and apparatus conventional in the art, for example using a Malvern Mastersizer 3000 (Worcestershire, UK) connected to a Hydro MV, wet dispersion unit (Malvern, Worcestershire, UK) as set out in the examples herein.

The aqueous phase (i.e. continuous phase) of the gelled oil-in-water emulsion may constitute from 60 to 99 wt. %, preferably from 70 to 95 wt. %, for example from 75 to 90 wt. %, from 75 to 85 wt. %, or from 75 to 80 wt. % of the composition.

In addition to water, the pectin gelling agent and the emulsifying agent, other physiologically tolerable materials may also be present in the aqueous phase, for example, pH modifiers (e.g. buffering agents), sweeteners, bulking agents (i.e. fillers), anti-oxidants, aromas, flavouring agents, and colouring agents. The nature and concentration of any such materials may readily be determined by those skilled in the art.

The presence of bulking agents (i.e. fillers) in the aqueous phase aids in reducing water activity and thus in reducing microbial growth. Water activity may, for example, be reduced to below about 0.8, for example in the range 0.5 to 0.8, or 0.6 to 0.75, or 0.65 to 0.75. The amount and type of bulking agents may readily be selected by those skilled in the art. Suitable examples include, but are not limited to, sugar alcohols, sugars and mixtures thereof. Suitable sugar alcohols include sorbitol and xylitol and mixtures thereof. Sugars which may be used include trehalose, sucrose, glycerol and mixtures thereof. Bulking agents may constitute from 45 to 70 wt. %, preferably 50 to 65 wt. %, e.g. 55 to 60 wt. %, based on the aqueous phase. In some cases, the selected bulking agent(s) may also act as sweetening agents depending on their concentration. For example, the compositions according to the invention may contain xylitol, e.g. as 0.5 to 50 wt. %, preferably 1 to 40 wt. %, e.g. 15 to 40 wt. %, in order to improve taste.

The presence of bulking agents, such as sugar alcohols, in the gelled aqueous phase leads to a low water activity. The emulsion according to the invention has a water activity in the range of about 0.4 to about 0.9. In some embodiments, the emulsion has a water activity in the range of about 0.5 to about 0.8.

Where a sweetener is included in the aqueous phase, this will typically be selected from natural sweeteners such as sucrose, fructose, glucose, reduced glucose, maltose, xylitol, maltitol, sorbitol, mannitol, lactitol, isomalt, erythritol, polyglycitol, polyglucitol, glycerol and stevia, and artificial sweeteners such as aspartame, acesulfame-K, neotame, saccharine, and sucralose. The use of non-cariogenic sweeteners is preferred.

Flavoring agents may be present in the compositions and may, for example, aid in taste masking certain lipids such as those which contain omega-3 fatty acids. Suitable flavors include, but are not limited to, citrus flavors, for example orange, lemon or lime oil.

The aqueous phase of the gelled emulsion has a low pH in the range from 3 to 5.5. In one set of embodiments, the pH of the aqueous phase may be in the range from 3.5 to 5, preferably 3.75 to 4.75, for example 3.75, 3.8, 3.85, 3.9, 3.95, 4, 4.05, 4.1, 4.15, 4.2, 4.25, 4.3, 4.35, 4.4, 4.45, 4.5, 4.55, 4.6, 4.65, 4.7 or 4.75. pH may be adjusted by the selection of suitable pH modifiers. pH modifiers may readily be selected by those skilled in the art and include food grade acids such as citric acid or malic acid. Buffering agents may also be used to adjust pH and include organic acid salt/organic acid buffering systems. Suitable buffering agents are well known in the art and include, for example, trisodium citrate/citric acid, trisodium citrate/malic acid, etc.

Where antioxidants are present in the aqueous phase these will be water soluble and include, for example, ascorbic acid, citric acid and salts thereof such as sodium ascorbate. Depending on the choice of oil, these may be supplied in a form which contains an antioxidant such as vitamin E, for example. If present, the amount of any anti-oxidant(s) may be up to 3 wt. % of the overall formulation, e.g. up to 1 wt. %.

In addition to the lipid(s), the oil phase of the emulsion may also if desired contain physiologically tolerable lipid soluble materials, for example pharmaceutically acceptable agents, anti-oxidants (e.g. vitamin E), flavorings, and coloring agents.

In some embodiments, additional physiologically active agents may also be present in the gelled emulsions herein described. These may be provided in the aqueous and/or oil phases and may be dissolved and/or dispersed in one or both of these phases. Other actives which may be present in the oil phase include fat soluble active agents.

In one embodiment, the gelled oil-in-water emulsions according to the invention may comprise, consist essentially of, or consist of, the following components:
(a) water;
(b) low-methoxy amidated pectin;
(c) one or more physiologically tolerable lipids;
(d) an emulsifying agent which is a phospholipid or mixture of phospholipids;
(e) one or more bulking agents;
(f) one or more pH modifiers;
(g) one or more flavour or colouring agents; and
(h) optionally one or more additional physiologically active agents, for example one or more pharmaceutically active agents or nutraceuticals.

By “consisting essentially of” it is intended that the emulsions will be substantially free from (e.g. free from) other components which materially affect their properties. By “consists of” it is intended that the emulsions will be substantially free (e.g. free from) from any other components than those listed.

The compositions of the invention are provided in the form of a dose unit. By “dose unit” it is intended that the composition will be taken orally by the subject (e.g. administered to a patient) “as received”, i.e. it will not be broken or cut before oral delivery. The weight of the dose unit will therefore be such that the composition is suitable for delivery in this way. For example, it may have an overall weight in the range from 50 to 3,000 mg, e.g. 250 to 3,000 mg or 500 to 2,500 mg, especially 100 to 2,000 mg, e.g. 750 to 2,000 mg, particularly 100 to 1,500 mg, more particularly 400 to 1,500 mg, more especially 400 to 1,000 mg.

In one set of embodiments, the dose units will generally be quite large, e.g. having a mass of from 400 to 3,000 mg, e.g. 600 to 1,500 mg. The overall dose unit weight may be selected as required. For example, it may be scaled up or down, dependent on the nature of the selected active components and their intended dose.

Each dose unit will consist of a self-supporting, gelled oil-in-water emulsion as herein described. As will be understood, in this case the dose unit will contain only the defined oil and aqueous phases, i.e. it will be free from any other components. Individual dose units may be prepared from a larger piece of the gelled emulsion which is divided, e.g. by cutting. More typically, however, each dose unit will be formed by extrusion or moulding from a liquid emulsion, or incompletely gelled emulsion, prior to gelation (i.e. above the gelling temperature of the pectin gelling agent).

Alternatively, a core of the gelled oil-in-water emulsion may be provided with a suitable coating of a physiologically tolerable coating material. Such coatings may be of the type conventional in the pharmaceutical and nutraceutical industry and may be applied by any conventional means, for example by dipping or spraying. In one set of embodiments, the gelled oil-in-water emulsions herein described may therefore be provided with a coating. For example, these may be provided within a capsule shell which dissolves in the mouth.

Viewed from another aspect the invention thus provides an orally administrable capsule comprising a capsule shell enclosing a gelled oil-in-water emulsion as herein described.

In the capsules of the invention, the shell may be of any physiologically tolerable material but will typically be a sugar, a biopolymer or a synthetic or semi-synthetic polymer which is soluble or disintegrable in saliva or fluid within the gastrointestinal tract. The shell may be soft, but is preferably substantially rigid. Particularly desirably, the capsules will have the consistency of a “jelly bean”. The shell will preferably be of a material and a thickness to prevent oxidation of the contents. The shell may comprise a sugar or cellulose, for example sorbitol. The use of sugars and cellulose as capsule shell materials is well-known in the pharmaceutical and nutraceutical fields.

The capsule shell material may thus typically be a sugar, e.g. sucrose, fructose, maltose, xylitol, maltitol or sorbitol, but may additionally contain hydrocolloid materials such as for example carageenan, alginate, pectin, cellulose, modified cellulose, starch, modified starch, gum arabic, etc. The capsule shell may contain other ingredients such as, for example, artificial sweeteners, colors, fillers, flavors, antioxidants, etc.

The capsule shell may be pre-formed such that the oil-in-water emulsion can be filled into the shell either as a liquid, or once set. Alternatively a shell precursor (e.g. a solution) may be coated onto the set emulsion, for example using standard coating techniques. If desired the capsule may be further coated, e.g. with a wax.

Preparation of the gelled oil-in-water emulsions herein described may be carried out by emulsification of the aqueous and oil phase components. It will be understood that emulsification is carried out under conditions in which the aqueous phase is a liquid (for example a viscous liquid), i.e. prior to the formation of a gel. Emulsification will thus be carried out at a temperature above the sol-gel transition temperature of the pectin gelling agent. Subsequent cooling of the emulsion below its sol-gel temperature results in the desired gelled emulsion.

Prior to emulsification, any selected active agents may be added to the oil and/or aqueous phase of the composition. This may be done, for example, by dissolving or dispersing the active in the selected oil or in the aqueous phase prior to forming the emulsion.

Alternatively, the selected active agent(s) may be added to a mixture of the aqueous and oil phase components prior to emulsification. During the emulsification process, the active agents will typically migrate to the oil or aqueous phase depending on their hydrophilic/lipophilic characteristics.

Emulsion formation may be effected by conventional techniques and using known equipment, for example a homogenizer based on the rotor-stator principle. The speed and duration of stirring may be adjusted as required, for example it may be varied to achieve the desired shearing force to provide the desired droplet size.

Emulsification will generally be carried out under a controlled atmosphere in order to avoid oxidative degradation of the lipid and/or any active agents. For example, emulsification may be carried out in the presence of a non-oxidising gas such as nitrogen. De-gassing to remove air bubbles may also be carried during the production process, for example prior to mixing the components of the emulsion, once the liquid emulsion has been formed, prior to packaging of the set emulsion, etc. De-gassing may be carried out using any conventional means such as the application of a vacuum, or sparging with a non-oxidising gas (e.g. nitrogen).

After emulsification and gelling, the emulsion may be dried to reduce the water content. If dried, however, it will still retain a continuous gelled aqueous phase as herein described and a water content within the limits herein defined.

The gelled oil-in-water emulsions are provided in dose unit form as herein described. Individual dose units may be formed by methods such as molding, extrusion or cutting. Typically, however, the dose units may be formed by filling of the liquid emulsion into molds, e.g. the individual molds of a blister pack which is then sealed. The dose units will typically be in tablet or lozenge form.

Methods for preparation of the gelled oil-in-water emulsions herein described form a further aspect of the invention. Viewed from a further aspect, the invention thus provides a method for preparing an orally administrable, gelled oil-in-water emulsion as herein described, said method comprising: forming an oil phase which comprises one or more physiologically tolerable lipids; forming an aqueous phase comprising a gelling agent which is low-methoxy amidated pectin; combining said oil phase and said aqueous phase in the presence of an emulsifier which is a phospholipid or mixture of phospholipids to form an oil-in-water emulsion; and allowing said emulsion to gel. Optionally, prior to or after allowing the emulsion to gel, the emulsion may be divided into individual dose units.

The dose units are preferably individually packaged in air-tight containers, e.g. a sealed wrapper or more preferably a blister of a blister pack. In another aspect, the invention thus provides a package comprising an air-tight and light-tight compartment containing one dose unit of a composition according to the invention. By excluding both air (i.e. oxygen) and light from the packaged dose unit, long term stability of the active components is enhanced.

The packages according to the invention are preferably provided in the form of blister packs containing at least two dose units, e.g. 2 to 100, preferably 6 to 30 dose units. The blister pack will generally comprise a metal, metal/plastic laminate or plastic sheet base having molded indentations in which the dosage form is placed. The pack is normally sealed with a foil, generally a metal or a metal/plastic laminate foil, for example by applying heat and/or pressure to the areas between the indentations. The use of a metal or metal/plastic laminate to form the blister pack serves to prevent air (i.e. oxygen), light and humidity from penetrating the contents of the blister pack thus enhancing the stability of active component(s). The packages according to the invention are preferably filled under a non-oxidising gas atmosphere (e.g. nitrogen) or are flushed with such a gas before sealing.

The gelled oil-in-water emulsions according to the invention find use both as pharmaceuticals, i.e. for therapeutic purposes, and as nutraceuticals to maintain or augment health and/or general well-being of a human or animal subject. For this purpose, it is intended that they are taken orally, lightly chewed in the mouth and then swallowed. It is not intended that they should remain in the mouth or need to be chewed for an extended period. Due to their soft texture, light chewing is sufficient to fragment the dosage form into smaller pieces which are easily swallowed.

When used as nutraceuticals, for example, the compositions herein described may be used as a supplement (e.g. as a dietary supplement) for maintaining the general health and/or well-being of a subject. Any agent known for its nutraceutical effects may be provided in the compositions and suitable agents are well known in the art. Suitable nutraceuticals include, but are not limited to, any of the following: essential fatty acids (e.g. mono and poly-unsaturated fatty acids), essential amino acids (e.g. taurine, tryptophan, tyrosine, cysteine and homocysteine), vitamins (e.g. vitamins A, B1-B12, C, D, E and K), minerals (e.g. iodine, selenium, iron, zinc, calcium and magnesium), flavonoids, carotenoids (e.g. beta carotene, alpha carotene, luteine, zeoxantaine, xanthophylls and lycopene), phytosterols, sapponins, probiotics, dietary fibres (e.g. insoluble fibre and beta-glucans), and plant extracts (e.g. aloe vera, evening primrose oil, garlic, ginger, ginseng, green tea, caffeine and cannabinoids). Where magnesium or calcium are present, these will generally be used in the form of their phosphate salts.

The Applicant has found that the compositions herein described allow for the delivery of certain nutraceutical components that may be incompatible with gelatin-based emulsions such as plant extracts that contain polyphenols. In particular, the compositions may contain one or more of the following components: proanthocyanidins (e.g. cranberry extract); rosmarinic acid (e.g. from lemon balm); catechins (e.g. green tea extract); anthocyanin-based extracts (e.g. derived from bilberry, blueberry, elderberry, chokeberry);

carotenoids; phytochemicals (e.g. caffeic acid, (−)-catechin, chlorogenic acid, ferulic acid, gallic acid, (−)-epigallocatechin, rutin, and trans-cinnamic acid).

In particular, the gelled oil-in-water emulsions herein described may be used as a source of one or more essential fatty acids, such as PUFAs or their esters, e.g. omega-3, omega-6 and/or omega-9 fatty acids and their ester derivatives. Examples of omega-3 acids include α-linolenic acid (ALA), stearidonic acid (SDA), eicosatrienoic acid (ETE), eicosatetraenoic acid (ETA), eicosapentaenoic acid (EPA), docosapentaenoic acid (DPA), docosahexaenoic acid (DHA), tetracosapentaenoic acid and tetracosahexaenoic acid. Examples of omega-6 acids include linoleic acid, gamma-linolenic acid, eicosadienoic acid, dihomo-gamma-linolenic acid (DGLA), arachidonic acid (AA), docosadienoic acid, adrenic acid, docosapentaenoic acid, and calendic acid. Examples of omega-9 acids include oleic acid, eicosenoic acid, mead acid, erucic acid and nervonic acid. Omega-3 acids are especially preferred, particularly EPA and DHA.

The health benefits of essential fatty acids, in particular omega-3 fatty acids, are well known. For example, these may lower triglyceride levels and/or lower cholesterol levels. Omega-3 fatty acids are vital to everyday life and health. The beneficial effects of EPA and DHA on lowering serum triglycerides are well known. They are also known for other health benefits such as cardio-protective effects, e.g. in preventing cardiac arrhythmias, stabilising atherosclerotic plaques, reducing platelet aggregation, and reducing blood pressure. They find use therefore in treating and/or preventing vascular disease. Other benefits of omega-3 fatty acids include the prevention and/or treatment of inflammation and neurodegenerative diseases, and improved cognitive development and function.

The essential fatty acids may form part or the whole of the oil phase in the gelled emulsion, preferably at least 10% wt, more especially at least 50% wt, particularly at least 80% wt. of that phase. They may be used as single compounds or as compound mixtures, e.g. plant or marine oils. The free fatty acids, the monoacyl glycerides and diacylglycerides may be prepared by full or partial hydrolysis of triacylglycerides, for example acid, base, or enzyme-catalysed hydrolysis, e.g. using lipases such as pancreatic lipases and/or lipases which may be produced from bacteria as fermentation products. Alkyl esters of essential fatty acids may be prepared by transesterification using the appropriate alkanol or by esterification of the free fatty acid with that alkanol. Where a free fatty acid is used, this may be in acid form or salt form (e.g. wholly or partially in salt form), and preferably constitutes 5 to 75% wt, especially 10 to 35% wt. of the essential fatty acid in the oil phase. Salt forms may be preferred.

The gelled oil-in-water emulsions herein described also find use as pharmaceuticals in the treatment or prevention of a range of medical conditions which are responsive to the chosen active agent(s). As will be appreciated, the nature of such conditions will be dependent on the selected active agent(s), but can readily be determined by those skilled in the art.

Any drug substance having a desirable therapeutic and/or prophylactic effect may be used. This includes drug substances which are lipophilic or hydrophilic. Classes of suitable drug substances include, but are not limited to, any of the following: analgesics; anti-inflammatories; anti-cancer agents; cardiovascular agents; biological agents; anti-allergy agents (e.g. antihistamines); decongestants; anti-nausea agents, drugs affecting gastrointestinal function; drugs acting on the blood and blood-forming organs; drugs affecting renal and cardiovascular function; anti-fungal agents; urological agents; hormones; antimicrobial agents, anti-epileptical agents; psycholeptical agents; antipsychotic agents; psychoanaleptical agents; anticholinesterase agents; and carotenoids.

The quantity of drug substance per unit dose of the compositions of the invention will conveniently be in the range of 10 to 100% of the recommended daily dose for an adult or child.

Viewed from another aspect, the invention thus provides a gelled oil-in-water emulsion as herein described for use in therapy.

Viewed from still another aspect, the invention provides a gelled oil-in-water emulsion as herein described which contains at least one pharmaceutically active component for oral use in the treatment of a condition responsive to said pharmaceutically active component.

In another aspect the invention provides the use of a pharmaceutically active component in the manufacture of a medicament for oral use in the treatment of a condition responsive to said pharmaceutically active component, wherein said medicament is provided in the form of a gelled oil-in-water emulsion as herein described.

Corresponding methods of medical treatment form a further aspect of the invention. Viewed from a yet further aspect, the invention thus provides a method of treatment of a human or non-human animal subject (e.g. a patient) to combat a condition responsive to a pharmaceutically active agent, said method comprising the step of orally administering to said subject a pharmaceutically effective amount of said agent in the form of a gelled oil-in-water emulsion as herein described.

In another aspect the invention provides the use of a gelled oil-in-water emulsion as herein described as a nutraceutical. Corresponding methods of administering the gelled oil-in-water emulsion in order to achieve a nutraceutical effect also form part of the invention.

Viewed from another aspect the invention thus provides a method of administering an active agent to a human or non-human animal subject to enhance and/or maintain said subject's health or wellbeing, said method comprising the step of orally administering to said subject a nutraceutically effective amount of an active agent in the form of a gelled oil-in-water emulsion as herein described.

In another aspect the invention provides the use of a gelled oil-in-water emulsion as herein described as a nutraceutical.

When used in any of the above treatments or methods, or as nutraceutical supplements or pharmaceutical formulations, an effective amount of the active agent can readily be determined.

The effective dose level for any particular subject will depend on a variety of factors including the disorder and its severity, the identity and activity of the particular composition, the age, bodyweight, general health of the subject (e.g. patient), timing of administration, duration of treatment, other drugs being used in combination with the treatment, etc. It is well within the skill of those in the art to select the desired dose to achieve the desired therapeutic effect.

The invention will now be described further with reference to the following non-limiting Examples.

### EXAMPLES

### Test Methods:

### 1. Small Scale Deformation

Rheological analyses on the gels were performed with a rheometer (Malvern Kinexus ultra+, Westborough, United States). The lower plate was a standard 60 mm stainless steel plate, whereas the upper geometry was a CP4/40 40 mm diameter 4° angle cone. Instrument calibration (zero gap) was performed prior to analysis. After gel preparation, approximately 2 grams of gel was placed on the lower plate, which was heated up to 60° C. The rheometer was operated in 0.1% shear strain controlled mode and the frequency was set to 1 Hz. The chosen strain was confirmed to be within the linear viscoelastic region for all samples. In order to avoid evaporation, the pectin emulsion gel samples were covered with silicone oil (10 cS fluid, Dow Corning, UK) prior to measurement. The viscoelastic properties of the sample were obtained by using a temperature gradient of 2° C./min, with a start and end temperature at 60° C. and a holding time of 15 min at 20° C. The results were analyzed using rSpace for Kinexus software. The gelling and melting temperatures of the samples were estimated as the temperature at which the phase angle corresponded to 45° in the cooling and heating process, respectively. The maximum storage modulus (G′) (Pa) was determined as the highest measurement point during curing at 20° C.

### 2. Large Scale Deformation

Texture properties of the gels were analysed with TA.XT plusC Texture Analyser (Stable Micro Systems Ltd., UK). Upon preparation, the gels were cast using cylindrical molds of standard dimensions (19.6 mm height, 8 mm diameter). The gels were cured at ambient temperature for 18 hours prior to analysis. Single compression analysis and the standard texture profile analysis (TPA) were performed using a 5 kg load cell. A P/35 35 mm diameter cylinder aluminum probe supplied by Stable Micro Systems Ltd. was used. For the 75% strain single compression, pre-test and post-test speeds were 2 mm/sec, while the test speed was 0.5 mm/sec and the trigger force was 5 grams. Strain height was measured automatically during compression. Max stress (g) and strain at failure (%) data was obtained from the fraction moment of the gels. Gradient (N/m) was calculated by the ratio of force at 2% and 3% strain.

### 3. Water Activity Measurements

Water activity was measured with HygroPalm HC2-AW (Rotronic, Switzerland) at ambient temperature. The sample was placed into the measurement chamber and the water activity was recorded after 45 minutes.

### 4. Droplet Size and Size Distribution

Droplet sizes and size distributions were measured using a Malvern Mastersizer 3000 (Worcestershire, UK) connected to a Hydro MV, wet dispersion unit (Malvern, Worcestershire, UK). Analysis of the data was performed using the manufacturer's software (Mastersizer 3000, v1.0.1). Testing was carried out by dissolving and diluting the gelled emulsion in a 10% (v/v) HCl solution (1:100) at 50° C. The refractive index of water and corn oil was set to 1.33 (solvent) and 1.47 (dispersed phase), respectively, and the absorption index of the dispersed droplets set to 0.01. To avoid multiple scattering or low intensity of the scattered light, each dissolved emulsion was added to the dispersion unit (containing ˜125 ml water), until an obscuration of approximately 10% was obtained.

### 5. Viscosity

Viscosity of liquid emulsions above the sol/gel transition temperature was measured at 60° C. using the same equipment as in the rheological analysis (small scale deformation). Tests were conducted using a 40 mm 4° cone probe with first a sweep from 0.1 to 10 s⁻¹, immediately followed by a sweep from 10 to 0.1 s⁻¹.

### 6. pH Measurement

pH of the gels was measured using a spear pH meter (pH Spear™, Oakton, Germany) calibrated at 20° C., at ambient temperature.

### Example 1—Typical Gelled Oil-In-Water Compositions

### Materials:

| Component | Wt. % |
|---|---|
| Low-methoxy amidated pectin | 1.0-3.0 |
| Lecithin | 0.1-0.25 |
| Xylitol | 20-35 |
| Sorbitol | 10-20 |
| Malic acid | 0.5-2.5 |
| Trisodium citrate | 0.25-3 |
| Tricalcium citrate | 0-1.5 |
| Oil | 10-30 |
| Flavour | 0.05-3 |
| Colour | 0.001-3 |
| Ascorbic acid (vitamin C) | 0-0.75 |
| Water | To 100 |

### Method of Preparation:

1. Add water to a glass bottle and heat up to 70° C. on a water bath.
2. Dry mix the pectin and sorbitol and add to the pre-heated water. Mix with magnetic stirring for 10 minutes.
3. Increase the water bath temperature to 75° C.
4. Dry mix xylitol, trisodium citrate, malic acid, ascorbic acid and tricalcium citrate (optional), and add 1/3 of this dry mix each time (5+5+10 mins). Magnetic stirring for a total of 20 mins at 75° C.
5. Reduce the water bath temperature to 60° C.
6. Add lecithin to the water phase and mix for 10 min.
7. Mix the flavours with the oil at room temperature.
8. Add the oil and flavour mixture gradually. Homogenise at 60° C. for 6 min at 7.6 k rpm speed with homogenizer IKA T18 Ultra Turrax.

### Example 2—Large Scale Production

### Materials:

| | Weight | Weight |
|---|---|---|
| Component | (kg) | (%) |
| Water | 83.75 | 25.00 |
| Pectin | 10.39 | 3.10 |
| Sorbitol | 47.47 | 14.17 |
| Xylitol | 95.99 | 28.65 |
| Stevia | 0.17 | 0.05 |
| Trisodium Citrate | 6.87 | 2.05 |
| Malic acid | 5.19 | 1.55 |
| Vitamin C | 1.12 | 0.33 |
| Tricalcium citrate | 0.80 | 0.24 |
| Lecithin | 0.84 | 0.25 |
| Aroma & Colour | 3.90 | 1.16 |
| Vegetable ω3 oil | 78.56 | 23.45 |
| Total | 335 | 100 |

### Production Method:

83.75 kg of purified water is placed in a heated, mixer equipped heating vessel, and heated to 70° C. 10.39 kg of pectin together with 47.47 kg of sorbitol is added.

The content is heated with stirring up until 75° C. 48.0 kg of xylitol (portion 1 of 2) is added and stirred for 15 minutes and until the temperature has reached 65° C.

The remaining 46.0 kg of xylitol is added and stirred for an additional 20 minutes and until temperature has reached 65° C.

A premix containing 1.99 kg xylitol, 0.17 kg stevia, 6.87 kg trisodium citrate, 5.19 kg malic acid, 1.12 kg ascorbic acid, and 0.80 kg tricalcium citrate is then added under vigorous mixing for 2 minutes and is then stirred for an additional 15 minutes until the temperature has reached 70° C.

The content is deaerated by use of vacuum for 5 minutes. The temperature is adjusted to 60° C.

0.84 kg of lecithin is added under vigorous mixing and stirred for 2 minutes.

3.9 kg of a premix with the aroma components and colorant is added through a side valve of the vessel.

78.56 kg of oil is gradually added under continuous stirring, and the content is stirred until the mixture has reached at least 60° C.

The content is then homogenized for 300 seconds by use of a rotor/stator High Shear Mixer.

The content is deaerated by use of vacuum for 120 seconds. The temperature is adjusted to 60° C.

The liquid mass is filled into aluminum blister molds, preferably under nitrogen.

### Example 3—Testing-Droplet Size

Pectin emulsions were prepared in accordance with the method of Example 1. These had varying pH and calcium concentrations but a constant pectin concentration of 3.10 wt. %. This concentration is based on the raw material as supplied, i.e. standardised with sucrose. The actual concentration of pectin is expected to be about 60% of this value, i.e. about 1.86 wt. %. The emulsion compositions are set out in Table 1:

TABLE 1
Composition of Emulsions

| | 1b | 2b | 3b | 4b | 5b | 6b | 7b |
|---|---|---|---|---|---|---|---|
| pH | 3.80 | 4.08 | 4.20 | 4.30 | 4.40 | 4.50 | 4.70 |
| Ca | 0 | 50 mM | 75 mM | 100 mM | 125 mM | 150 mM | 175 mM |
| Water | 25.10% | 25.10% | 25.10% | 25.10% | 25.10% | 25.10% | 25.10% |
| Pectin¹ | 3.10% | 3.10% | 3.10% | 3.10% | 3.10% | 3.10% | 3.10% |
| Sorbitol | 14.17% | 14.17% | 14.17% | 14.17% | 14.17% | 14.17% | 14.17% |
| Xylitol | 28.42% | 28.70% | 28.72% | 28.73% | 28.72% | 28.71% | 28.70% |
| Trisodium | 2.05% | 2.05% | 2.05% | 2.05% | 2.05% | 2.05% | 2.05% |
| citrate | | | | | | | |
| Malic acid | 2.07% | 1.55% | 1.41% | 1.28% | 1.17% | 1.06% | 0.95% |
| Ascorbic acid | 0.34% | 0.34% | 0.34% | 0.34% | 0.34% | 0.34% | 0.34% |
| Tricalcium | 0 | 0.24% | 0.36% | 0.48% | 0.60% | 0.72% | 0.84% |
| citrate | | | | | | | |
| Lecithin² | 0.25% | 0.25% | 0.25% | 0.25% | 0.25% | 0.25% | 0.25% |
| Lemon flavour | 0.52% | 0.52% | 0.52% | 0.52% | 0.52% | 0.52% | 0.52% |
| Orange flavour | 0.52% | 0.52% | 0.52% | 0.52% | 0.52% | 0.52% | 0.52% |
| Sunflower oil | 23.46% | 23.46% | 23.46% | 23.46% | 23.46% | 23.46% | 23.46% |
| Total | 100.00% | 100.00% | 100.00% | 100.00% | 100.00% | 100.00% | 100.00% |

All % are given as “% by weight” (based on the total weight of the emulsion)
¹GENU ™ pectin type LM-104 AS-FS, CP Kelco (E440 amidated pectin standardised with sucrose). Pectin concentration is based on the raw material.
²GIRALEC HE-60 enzymatically hydrolysed sunflower lecithin, Lasenor(E-322).

The following pectin emulsions were also prepared:

“1b more malic”: Corresponding to emulsion 1b except malic acid was increased to 2.4 wt. % (with a corresponding reduction in wt. % of xylitol)

“2b no flavour”: Corresponding to emulsion 2b except the flavours were replaced with more sunflower oil.

Each emulsion was prepared according to the general method set out in Example 1 and placed in 50 g or 65 g cylinders for testing. Droplet sizes were measured as set out herein. Average droplet sizes for the different emulsions at T=0 hours are shown in Table 2:

TABLE 2
Droplet Size

| | D [4;3] | D [3;2] |
|---|---|---|
| | (μm) | (μm) |
| 1b | 16.9 | 5.8 |
| 1b more malic | 26.5 | 8.17 |
| 2b | 24.8 | 7.71 |
| 2b no flavour | 16 | 5.57 |
| 3b | 35.4 | 9.93 |
| 4b | 38.5 | 9.95 |
| 5b | 41.7 | 10.7 |
| 6b | 35.8 | 9.28 |
| 7b | 32.7 | 8.63 |

### Example 4—Testing-Rheology and pH

Tests were carried out on the gelled emulsions produced in Example 3 according to the small scale deformation method described herein. Tₘ and T_{g} were determined as the temperature at which phase angle goes above or drops below 45°, respectively, under the given temperature gradient, strain and frequency. The pH of the emulsion gels was measured after they were set at ambient room temperature using a spear pH meter (pH Spear™, Oakton, Germany) calibrated at 20° C. The results are set out in Table 3:

TABLE 3
Small scale deformation

| | G′ max | | | |
|---|---|---|---|---|
| | (Pa) | | | |
| | (after 15 | Tm | Tg | |
| Gel | mins @ 20° C.) | (° C.) | (° C.) | pH |
| 1b | 2822 | 51.5 | 36 | 3.94 |
| 1b more malic | 3638 | 65 | 41 | 3.87 |
| 2b | 4571 | 58 | 41 | 4.17 |
| 2b no flavour | 4129 | 57 | 41 | 4.15 |
| 3b | 4743 | 59 | 43 | 4.22 |
| 4b | 4409 | 58 | 43 | 4.33 |
| 5b | 4446 | 59 | 44 | 4.40 |
| 6b | 3832 | 58 | 43 | 4.54 |
| 7b | 3336 | 57 | 41 | 4.69 |

The high Tm value is notable and leads to long term stability of the product in warmer climates.

### Example 5—Testing-Viscosity

Viscosity values were measured at 60° C. in respect of the gelled emulsions produced in Example 3 according to the method described herein. The results are shown in Table 4:

TABLE 4
Viscosity

| | First viscosity | | | Second viscosity | | |
|---|---|---|---|---|---|---|
| | (0.1-10 s⁻¹) | | | (10-0.1 s⁻¹) | | |
| Gel | 0.1/s | 1/s | 10 /s | 0.1/s | 1/s | 10/s |
| 1b | 25.36 | 11.19 | 6.60 | 17.95 | 11.79 | 6.61 |
| 1b more malic | 57.56 | 19.22 | 5.96 | 26.76 | 14.95 | 5.98 |
| 2b | 22.71 | 13.14 | 7.90 | 21.72 | 14 | 7.79 |
| 2b no flavour | 26.99 | 15.38 | 7.93 | 23.22 | 15.18 | 7.4 |
| 3b | 24.30 | 14.51 | 8.57 | 22.68 | 15.49 | 8.41 |
| 4b | 24.44 | 14.60 | 8.75 | 22.96 | 15.72 | 8.75 |
| 5b | 23.89 | 14.41 | 8.72 | 22.75 | 15.59 | 8.70 |
| 6b | | | | 21.45 | 12.68 | 8.35 |
| 7b | 23.78 | 14.56 | 8.709 | 21.90 | 15.74 | 8.719 |

All systems are quite shear thinning, with some thixotropy. 1b and especially “1b more malic” show significantly increased thixotropy.

### Example 6—Testing-Texture/Large Scale Deformation

Tests were carried out on the gelled emulsions produced in Example 3 according to the large scale deformation methods described herein. The results are set out in Table 5:

TABLE 5
Texture/Large scale deformation

| | Force at break | Strain at break | Initial gradient |
|---|---|---|---|
| | (g) | (%) | (g/%) |
| 1b | 1483 | 51.7 | 2.203 |
| 2b | 1740 | 48.1 | 3.842 |
| 5b | 2094 | 50.6 | 3.620 |
| 7b | 2537 | 52.9 | 3.806 |

These results are similar to those for conventional gelatin-based emulsions. The gelled emulsions according to the invention can thus be expected to exhibit similar chew and mouthfeel characteristics to the gelatin-based products.

### Example 7—Gelled Oil-In-Water Composition

### Materials:

| | Weight per | |
|---|---|---|
| | unit dose | |
| | 1700 mg | Weight |
| Component | (mg) | (%) |
| Active Ingredients: | | |
| Algae oil containing DHA, EPA | 400 | 23.53 |
| Excipients: | | |
| Xylitol | 488 | 28.68 |
| Purified water | 425 | 25.00 |
| Sorbitol | 241 | 14.17 |
| Pectin E440¹ | 53 | 3.10 |
| Trisodium citrate | 35 | 2.05 |
| Malic acid | 26 | 1.55 |
| Lemon Flavour | 6.1 | 0.36 |
| Passion fruit flavor | 6.1 | 0.36 |
| Ascorbic acid | 5.7 | 0.34 |
| Natural orange flavor | 5.5 | 0.33 |
| Partially hydrolysed lecithin E322² | 4.3 | 0.25 |
| Tricalcium citrate | 4.0 | 0.24 |
| Steviol glycosides | 0.85 | 0.05 |
| Betacarotene 30% | 0.12 | 0.007 |

¹GENU ™ pectin type LM-104 AS-FS, CP Kelco (E440 amidated pectin standardised with sucrose).
²GIRALEC HE-60 enzymatically hydrolysed sunflower lecithin, Lasenor (E-322).

The composition was prepared similar to the method in Example 2. The liquid mass was filled into aluminum blister molds under nitrogen to produce unit dose forms each weighing 1700 mg.

After they were set at ambient temperature, the pH and water activity of the emulsion gels was measured according to the methods described herein. Measured pH was 4.01. Measured water activity was 0.732.

Testing was carried out using the methods detailed herein to obtain the data in Table 6:

TABLE 6
characterisation of gels

| | | | | Vis- | Vis- | Droplet | Droplet |
|---|---|---|---|---|---|---|---|
| | G′ max | Tm | Tg | cosity | cosity | size | size |
| | (Pa) | (° C.) | (° C.) | 1/s | 10/s | D [4; 3] | D [3; 2] |
| Batch 1 | | | | | | | |
| Pectin | 4039 | 54.3 | 40.3 | 13.6 | 7.7 | 15.5 | 3.99 |
| T = 0 | | | | | | | |
| Pectin | 4492 | 53.4 | 40.6 | 15.6 | 8.8 | 13.5 | 3.49 |
| 40° C. | | | | | | | |
| T = 30 days | | | | | | | |
| Pectin | 4560 | 53.6 | 40.9 | 15.5 | 8.9 | n/a | n/a |
| 40° C. | | | | | | | |
| T = 60 days | | | | | | | |
| Pectin | 5908 | 53.9 | 41.3 | 14.8 | 8.5 | 16.1 | 4.16 |
| 40° C. | | | | | | | |
| T = 90 days | | | | | | | |
| Pectin | 4981 | 54.5 | 41.5 | 15.2 | 9 | n/a | n/a |
| Zone II¹ | | | | | | | |
| T = 360 | | | | | | | |
| days | | | | | | | |
| Batch 2 | | | | | | | |
| Pectin | 4549 | 53.2 | 38.9 | 8.1 | 5.2 | 23.5 | 5.4 |
| algae oil | | | | | | | |
| T = 0 | | | | | | | |
| Pectin | 4596 | 53.9 | 40.3 | 10.0 | 5.01 | 19.5 | 5.01 |
| algae oil | | | | | | | |
| 40° C. | | | | | | | |
| T = 30 days | | | | | | | |
| Pectin | 5092 | 54.4 | 40.9 | 10.2 | 4.13 | 16.4 | 4.13 |
| algae oil | | | | | | | |
| 40° C. | | | | | | | |
| T = 60 days | | | | | | | |

¹ICH guideline Zone II, 25° C.

### Example 8—Comparison with Agar and Gelatin-Based Emulsion Gels

Emulsion gels using agar and pectin were prepared, characterized and compared to a traditional gelatin emulsion gel.

Agar gel (AG), Agar emulsion gel without oil (AWO), Agar emulsion gel (AEG), Pectin emulsion gel without oil (PWO), Pectin emulsion gel (PEG), and Gelatin emulsion gel (GEG) were prepared and tested as described in Baydin et al., Applied Food Research 2 (2022) 100225 (https://doi.org/10.1016/j.afres.2022.100225), the entire content of which is incorporated herein by reference. The composition of the gels is given in Table 7.

TABLE 7
Compositions of gels. All ingredients are given as percentage (w/w).

| | | Agar | | Pectin | | |
|---|---|---|---|---|---|---|
| | | emulsion | Agar | emulsion | Pectin | Gelatin |
| | | gel without | emulsion | gel without | emulsion | emulsion |
| | Agar gel | oil | gel | oil | gel | gel |
| Ingredient | (AG) | (AWO) | (AEG) | (PWO) | (PEG) | (GEG) |
| Water | 92.85 | 35.51 | 26.10 | 33.88 | 25.26 | 21.00 |
| Agar¹ | 7.15 | 2.72 | 2.00 | | | |
| Pectin² | — | — | — | 3.76 | 2.8 | — |
| Gelatin³ | — | — | — | — | — | 8.60 |
| Citrem | — | — | 1.50 | — | — | — |
| Xylitol | — | 38.64 | 28.40 | 38.10 | 28.4 | 28.40 |
| Sorbitol | — | 19.05 | 14.00 | 18.78 | 14 | 14.00 |
| MA⁴ | — | 1.36 | 1.00 | 2.01 | 1.5 | 1.00 |
| TCD⁵ | — | 2.72 | 2.00 | 2.68 | 2 | 2.00 |
| Ascorbic | — | — | — | 0.47 | 0.35 | — |
| acid | | | | | | |
| CCT⁶ | — | — | — | 0.32 | 0.24 | — |
| Lecithin⁷ | — | — | — | — | 0.45 | — |
| Corn oil | — | — | 25.00 | — | 25.00 | 25.00 |

¹Gelagar HDR 300 (B&V, Italy)
²GENU ™ pectin (LM-104 AS-FS) (CP Kelco, Denmark)
³Bovine gelatin (Type B 160 Bloom) (Gelita ®, Germany)
⁴Malic acid
⁵Trisodium citrate dihydrate
⁶Calcium citrate tetrahydrate
⁷Hydrolysed sunflower lecithin (Giralec HE-60) (Lasenor, Spain)

The gels were characterized and compared by rheological characterization, texture profile analyses, syneresis measurements, water activity measurements, droplet size analysis, in vitro lipolysis and microscopy as described in Baydin et al. (Applied Food Research 2 (2022) 100225), the entire content of which is incorporated herein by reference.

Of particular note are the following conclusions based on the results documented in Baydin et al., 2022:

For industrial production, PEG may be a more manufacturable formulation alternative to GEG since it has relatively similar Tₘ and T_{g} values to GEG and it did not exhibit large decreases in G′ₘₐₓ after re-melting.

Consumer acceptance of emulsion gels for oral delivery highly depend on their mechanical properties which describe their response to deformation, such as the chew in the mouth. GEG had the highest maximum stress among all gels, and it did not fail at 75% strain, pointing to the ductile texture of the gel. The polysaccharide emulsion gels were weaker and more brittle than GEG. However, compared to AEG, PEG had strain at failure and maximum force values closer to GEG, suggesting a more similar sensory perception.

Gelatin gels have a unique chewy texture that is familiar to consumers of gelatin-based jelly desserts, gummies, and confectionery. This texture is difficult to be replicated with polysaccharides since polysaccharide gels typically do not have a similar elastic/chewy texture. PEG had lower hardness than both AEG and GEG. However, it was less brittle and had higher resilience, cohesiveness, and springiness than AEG, potentially making its mouthfeel more similar to GEG.

### Example 9—Physical and Functional Properties of Plant-Based Pre-Emulsified Chewable Gels for the Oral Delivery of Nutraceuticals

Gelatin-based pre-emulsified chewable gels have presented advantages over traditional tablets, bulk oils, hard and soft capsules for oral delivery. Ethical, ecological, and religious considerations have increased the demand for plant-based gelling agents which can be formulated into chewable emulsion gels. Plant-based polysaccharide emulsion gels prepared with agar and pectin were compared to gelatin emulsion gels regarding rheological, textural, and functional properties. The agar emulsion gel had higher gelling/melting temperatures (T_{g}: 40° C., Tₘ: 90° C.) than the gelatin emulsion gel (T_{g}: 37° C., Tₘ: 45° C.), whereas pectin emulsion gel had a more similar gelling/melting profile to the gelatin formulation (T_{g}: 38° C., Tₘ: 54° C.). Texture analyses revealed that the agar emulsion gel had a harder and more brittle texture, whereas pectin emulsion gel had a softer texture than the gelatin emulsion gel. Pectin emulsion gels had the largest average droplet size (32 μm), followed by agar (13 μm) and gelatin emulsion gels (1 μm). The in vitro lipolysis experiments indicated that the polysaccharide emulsion gels were lipolyzed to a lower extent and had a slower initial lipolysis rate (agar: 2.8 μmol FFA/sec, pectin: 4.3 μmol FFA/sec), compared to the gelatin emulsion gel (24.9 μmol FFA/sec). The industrial potential and challenges of the polysaccharide emulsion gels were evaluated, and the results show that plant based pre-emulsified chewable gels can be manufactured for the oral delivery of nutraceuticals.

### INTRODUCTION

Food supplements and nutraceuticals are consumed to complement a diet with micronutrients, aiming to enhance health and provide medical benefits (DeFelice, 1995; Santini & Novellino, 2017). Essential fatty acids, vitamins, minerals, and carotenoids are among important nutraceuticals which may be incorporated into an applicable dosage form (Chen, Remondetto & Subirade, 2006; Karuna & Prasad, 2015). Typical oral delivery forms include soft and hard gel capsules, tablets, elixirs, syrups, and chewables (Adepu & Ramakrishna, 2021; Dille, Hattrem & Draget, 2018b). There has been an increasing demand for the development of user-friendly dosage forms, in which a chewable delivery system is considered to be a practical alternative (Dille, Hattrem & Draget, 2018a). The most common chewable delivery forms are similar to gummies/confectioneries in appearance, in which sweeteners, acidulants, aromas and colorants are used to make a well-tasting product. Besides having a good palatability, which is important for compliance, the product also has the uniformity of a tablet, and its chewable texture removes the need for water in the process of ingestion. Although chewable dosage forms offer a user-friendly design, their development and production have been challenging. In the formulation, the water-soluble actives are typically in a solubilized state, while fat-soluble actives need to be mixed throughout the continuous water phase as small oil droplets (Dille et al., 2018a). This may be especially challenging if the payload of the fat soluble active ingredient is high, as mixing of the oil phase requires a more complex production process, while maintaining the stability of the oil phase throughout the manufacturing process. The chemical stability of the embedded ingredient may to a certain extent be ensured by innovative formulation and packaging technology, while to obtain a stable droplet size at elevated temperatures for a longer period of time, the choice of emulsifier, and gelling agent is of high importance.

Emulsion gels i.e., gelled emulsions, are complex soft-solid colloidal materials which include an emulsion and a gel (Dickinson, 2012). Emulsion gels can be classified as emulsion droplet-filled gels and emulsion droplet-aggregated gels (Dickinson, 2012; Lin, Kelly & Miao, 2020b). In emulsion droplet-filled gels, a polysaccharide or protein gel constitutes the continuous gel matrix which contains embedded emulsion droplets (Gravelle & Marangoni, 2021). If the emulsion droplets strongly interact with the gel network, the droplets are generally referred to as active fillers. The term inactive filler is used for systems in which the emulsion droplets have little to no interaction with the surrounding gel network (Chen & Dickinson, 1999). In the context of oral delivery of nutraceuticals, emulsion gels are convenient delivery forms compared to traditional capsules, bulk oils or liquid emulsions which may present compliance issues for pediatric and geriatric populations, as well as patients suffering from dysphagia. In addition, emulsion gels provide physical stability and mechanical properties to emulsions (Lu, Mao, Hou, Miao & Gao, 2019).

Due to their unique texture and emulsifying capability, chewable gelatin-based emulsion gels have been studied, characterized, and patented (Dille et al., 2018a, 2018b; Hattrem, Dille, Seternes, Ege & Draget, 2018, 2015; Haug & Draget, 2007). Gelatin-based emulsion gels typically exhibit an active filler effect, whereas polysaccharide-based emulsion gels made with hydrocolloids with low surface activity typically exhibit an inactive filler effect (Dille et al., 2018a; Koç et al., 2019). One challenge with gelatin-based gels is the low melting temperature of gelatin which may result in the instability of the delivery system in warm climates during transportation or storage of the products (Baydin, Aarstad, Dille, Hattrem & Draget, 2022). Another disadvantage is the animal origin of gelatin, since it is obtained from the connective tissues of cattle, pigs, fish or poultry (Schrieber & Gareis, 2007). Ecological, ethical, and health concerns, religious constraints, as well as dietary restrictions may endorse vegetarian or vegan diets for individuals (Leitzmann, 2014). In the recent years, there has been an increase in the number of people who follow a plant-based diet (Alcorta, Porta, Tárrega, Alvarez & Vaquero, 2021). This has resulted in an increase in the demand for plant-based food alternatives in the market (Noguerol, Pagán, García-Segovia & Varela, 2021; Stannard, 2018).

Although gelatin is an animal-derived biopolymer, terms such as plant-based or “veggie gelatin” have been used to describe plant hydrocolloids with gelling properties (Alipal et al., 2021; Lestari, Octavianti, Jaswir & Hendri, 2019). Emulsion gels have been prepared with polysaccharides such as agar (Kim, Gohtani & Yamano, 1997, 1996, 1999; Yamano, Kagawa, Kim & Gohtani, 1996), pectin (Lupi et al., 2015), carrageenans (Fontes-Candia, Ström, Lopez-Sanchez, López-Rubio & Martínez-Sanz, 2020; Sala, de Wijk, van de Velde & van Aken, 2008), cellulose (Jiang et al., 2019), and other polysaccharides (Dun et al., 2020; Weiss, Scherze & Muschiolik, 2005; Yang et al., 2019). Although most plant-based hydrocolloids can act as stabilizing agents of emulsions due to structuring, thickening or gelling of the continuous phase, they usually lack the surface active properties to act as emulsifying agents (Dickinson, 2009). Therefore, an emulsifying agent is commonly included in combination with a plant-based gelling agent to provide sufficient emulsifying and stabilizing capacity in such systems (Banerjee & Bhattacharya, 2011; Shao et al., 2020). Another challenge with plant-based emulsion gels is their texture since gelatin has a characteristic elastic, chewy, melt-in-the-mouth texture, which is very difficult to mimic with plant-based alternatives (Karim & Bhat, 2008; Schrieber & Gareis, 2007). Other issues with polysaccharide emulsion gels have been reported, such as stability, syneresis, and the complexity of the systems (Lin, Kelly, Maidannyk & Miao, 2020a; Yue et al., 2022).

In addition to being more practical and user-friendly than traditional oral delivery forms, gelatin-based emulsion gels have been shown to have comparable stability and dissolution kinetics to a standard oral tablet (Dille et al., 2018b). Furthermore, the bioavailability of omega-3 polyunsaturated fatty acids eicosapentaenoic acid (EPA) and docosahexaenoic acid (DHA) significantly increased when orally delivered in a chewable gelatin-based emulsion gel compared to bulk oil (Haug et al., 2011). Digestibility of food emulsion gels are commonly studied through in vitro lipolysis experiments to preliminarily investigate the functionality of formulations. In vitro lipolysis studies of gelatin emulsion gels indicated the relation between the gel mesh size and speed of lipid digestion (Sarkar et al., 2015). In vitro lipolysis of agar emulsion gels was studied and compared with emulsions without agar present in the formulation, suggesting limitations of digestion due to entrapment of oil droplets in the agar network (Wang, Neves, Kobayashi, Uemura & Nakajima, 2013). Similarly, the lipolysis extent of pectin emulsion gels were lower compared to liquid emulsions (Yang et al., 2022). The methodology and digestive parameters of the in vitro lipolysis experiment may have a large impact on the results of the digestion studies (Li, Hu & McClements, 2011; Lin & Wright, 2018; Mella, Quilaqueo, Zúñiga & Troncoso, 2021). Therefore, for comparative analyses, it is of importance to test different formulations in a standardized in vitro lipolysis set-up (Li et al., 2011). To the authors' knowledge, a study comparing the extent of in vitro lipolysis of emulsion gels prepared with gelatin, agar, and pectin, has not been previously published.

The present study focuses on two polysaccharide-based emulsion gels: agar and pectin. Agars are moderately sulfated galactans, obtained from red seaweed, and are a mixture of agarose and agaropectin (Araki, 1956; Sousa, Rocha & Gonçalves, 2021). Pectins are a group of complex heteropolysaccharides, mainly containing galacturonic acid residues, and are obtained from terrestrial plants (Hua, Wang, Yang, Kang & Zhang, 2015). Commercial pectins may be amidated or methyl-esterified to varying degrees (Da Silva & Rao, 2006; Zeeb, Roth & Endreß, 2021). The scope of this study was to characterize emulsion gels made with polysaccharides, agar, and pectin, and to compare the rheological and textural properties of these gels with a traditional gelatin emulsion gel. In addition, agar and pectin gels that do not contain oil were compared to corresponding emulsion gels to study the impact of oil on the rheology and texture of the gels. Along with the comparison of the physical and textural properties of the gels, their functionalities were studied with gastrointestinal in vitro lipolysis experiments. An object of this investigation was to develop plant-based emulsion gels for the oral delivery of nutraceuticals and evaluate their potential as alternatives to gelatin-based emulsion gels.

### Materials and Methods

Materials. Agar (Gelagar HDR 300) was purchased from B&V, Italy. Bovine gelatin (Type B 160 Bloom, Batch #643208) was provided by Gelita®, Germany. Xylitol was provided by Danisco, UK. Sorbitol was purchased from Food Innovation, Norway. Malic acid (MA) and trisodium citrate dihydrate (TCD) were provided by MerckMillipore, USA. Corn oil (lot #MKCH1635) and ascorbic acid were purchased from Sigma, USA. Citrem (Grinsted N 12 veg kosher, batch #4011722438) was purchased from Danisco, Denmark. Hydrolyzed sunflower lecithin (Giralec HE-60) was provided by Lasenor, Spain. GENU® pectin (LM-104 ASFS) was provided by CP Kelco, Denmark. Calcium citrate tetrahydrate (CCT) was provided by Gadot Biochemical Industries, Israel. Bile extract porcine (lot #SLCC9272) was purchased from Sigma Aldrich, USA. Pancreatin from porcine pancreas (8×USP, lot #SLBZ5739) and lipase from porcine pancreas (100-500 U/mg, lot #SLBZ7254) were purchased from Sigma Aldrich, USA. Rabbit gastric lipase (RGE15, 15 U/mg lipase and 500 U/mg pepsin, lot #BCBV8659) was purchased from Lipolytech, France.

Composition and preparation of the gels. Agar gel (AG) was prepared by mixing agar and deionized water (18.2 MQcm Stakpure OmniaPure, Germany) at 90° C. for 30 min with magnetic stirring. After agar had completely dissolved, 0.06 (w/w) % Witafrol was added, and the solution was degassed using the Diaphragm Vacuum Pump (Vacuubrand, MZ 2C) until no air bubbles were visible. The water loss was compensated after degassing. The solution was allowed to gel at ambient temperature, i.e., an average of 22° C. laboratory environment. Agar emulsion gel without oil (AWO), i.e., agar gel with sugar alcohols and buffer salts, was prepared by first dry mixing agar, sorbitol, and xylitol. Ambient temperature water was added to the powder mixture and the contents were mixed at 90° C. for 30 min with magnetic stirring. The temperature was reduced to 65° C. and MA was added to the mixture and mixed for 10 min. Afterwards, TCD was added to the mixture and mixed for 10 min. Agar emulsion gel (AEG) was prepared in a similar way to AWO, with the exception of first adding TCD to the solution when the temperature was reduced to 65° C.

After TCD was fully dissolved, citrem was added to the mixture and mixed for 15 min with magnetic stirring. Afterwards, MA was added and mixed for 10 min. The water phase was degassed as described above. The temperature of the mixture was reduced to 50° C. and preheated corn oil at this temperature was included in the mixture. The water phase and the oil phase were homogenized at 50° C. using T18 digital ultra-Turrax® (IKA®, USA) at speed 9.8k rpm for 8 min.

Pectin emulsion gel without oil (PWO), i.e., pectin gel with sugar alcohols and buffer salts, was prepared by first dry mixing pectin and sorbitol, and then adding water and mixing with magnetic stirring at 75° C. for 25 min. Afterwards, xylitol, TCD, MA, ascorbic acid and CCT were dry mixed and gradually added to the mixture with a total mixing time of 20 min. The water phase was degassed as described above. Pectin emulsion gel (PEG) was prepared similarly to PWO. After degassing, the temperature was reduced to 60° C., preheated lecithin was added, and mixed for 20 min. Preheated corn oil was added and homogenized at 60° C. with 9.8k rpm for 8 min.

Gelatin emulsion gel (GEG) was prepared by mixing gelatin with water for 30 min with magnetic stirring at 60° C. Afterwards, sorbitol, xylitol, TCD and MA were added and mixed for 10 min after each addition. Preheated corn oil at 60° C. was added to the aqueous phase and homogenized at ambient temperature at 9.8k rpm speed for 5 min. The emulsion gel was degassed as described above.

The composition of the gels is given in Table 8. For AG, AWO and AEG, the ratio of agar to water was kept constant at 0.77. For PWO and PEG, pectin to water ratio was kept constant at 0.11.

TABLE 8
Abbreviations and compositions of the gels. All ingredient are given as weight percentage (w/w).

| | | Agar | Agar emulsion | Agar | Pectin emulsion | Pectin | Gelatin |
|---|---|---|---|---|---|---|---|
| | | Gel | gel without oil | emulsion | gel without oil | emulsion | emulsion |
| Ingredient | Function | (AG) | (AWO) | gel (AEG) | (PWO) | gel (PEG) | gel (GEG) |
| Water | Solvent | 92.8 | 35.51 | 26.10 | 33.88 | 25.26 | 21.00 |
| Agar | Gelling | 7.15 | 2.72 | 2.00 | — | — | — |
| Pectin | agent | — | — | — | 3.76 | 2.8 | — |
| Gelatin | | — | — | — | — | — | 8.60 |
| Citrem | Emulsifier | — | — | 1.50 | — | — | — |
| Xylitol | Sweeteners | — | 38.64 | 28.40 | 38.10 | 28.4 | 28.40 |
| Sorbitol | | — | 19.05 | 14.00 | 18.78 | 14 | 14.00 |
| MA | Buffer | — | 1.36 | 1.00 | 2.01 | 1.5 | 1.00 |
| TCD | Salts | — | 2.72 | 2.00 | 2.68 | 2 | 2.00 |
| Ascorbic Acid | | — | — | — | 0.47 | 0.35 | — |
| CCT | Calcium salt | — | — | — | 0.32 | 0.24 | — |
| Lecithin | Emulsifier | — | — | — | — | 0.45 | — |
| Corn Oil | Oil | — | — | 25.00 | — | 25.00 | 25.00 |

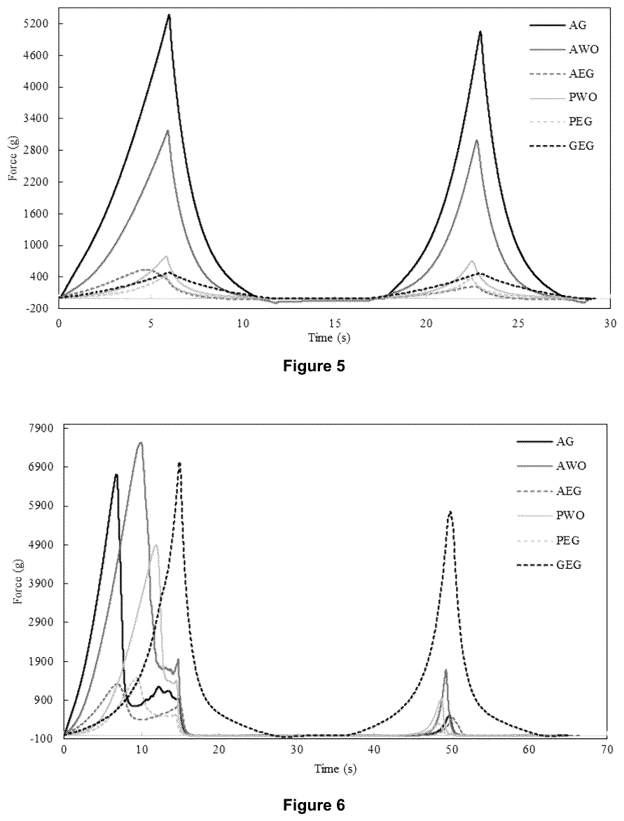

Rheological characterization. Small amplitude oscillatory shear (SAOS) measurements on the gels were performed with a rheometer (Malvern Kinexus ultra+, Westborough, United States). The upper geometry was serrated PP40X SW1648 SS for all agar gels, whereas the lower plate was serrated PLS40X S1586 SS for AG and KNX0127, curved sandblasted 50 mm for AWO and AEG. For PWO, PEG and GEG, the lower plate was PL61 ST S2579 SS, and the upper geometry was CP4/40 40 mm diameter 4° cone angle. A solvent trap was used for AG, PEG and GEG to prevent evaporation during the measurement. The protocol given in Baydin et al. (2022) was followed. Instrument calibration (zero gap) was performed prior to analysis. After gel preparation, approximately 2 g of gel was placed on the Peltier temperature controlled lower plate, which was heated up to 60° C. The rheometer was operated in 0.1% shear strain controlled mode and the frequency was set to 1 Hz. The chosen strain was confirmed to be within the linear viscoelastic region through a strain sweep performed on AG (one of the most brittle gels tested in this study) between 0.001 and 100% strain (FIG. 4). The viscoelastic properties of the sample were obtained by using a temperature gradient of 2° C./min, with a start and end temperature at 60° C. and a holding time of 15 min at 20° C. for PEG and GEG. For AG, AEG and AWO, the end temperature was 90° C. and oscillation continued for 10 min at 90° C. The results were analyzed using rSpace for Kinexus software (version 1.75) and Microsoft Excel (version 2201). The gelling temperature (T_{g}) and melting temperature (Tₘ) of the samples were estimated as the temperature at which the phase angle corresponded to 45° in the cooling and heating process, respectively. The maximum storage modulus (G′ₘₐₓ, Pa) was determined as the highest measurement point during setting at 20° C.

Texture profile analyses. Texture properties of the gels were analyzed with TA.XT plusC Texture Analyser (Stable Micro Systems Ltd., UK). Upon preparation, the gels were cast using cylindrical molds of standard dimensions (19.6 mm height, 8 mm diameter). The gels were set at ambient temperature for 24 h prior to analysis. Single compression analysis and standard texture profile analysis (TPA) were performed at ambient temperature, using a 5 kg load cell for AG, AWO, AEG, PEG and PWO, and a 30 kg load cell for GEG and 75% strain TPA test of AG. P/35 35 mm diameter cylinder aluminum probe supplied by Stable Micro Systems Ltd. was used for both single compression and TPA analyses. For the 75% strain single compression, pre-test and post-test speeds were 2 mm/s, while the test speed was 0.1 mm/s and the trigger force was 5 g. Strain height was measured automatically during compression. Max force (g) and strain at failure (%) data was obtained from the fraction moment of the gels. Young's modulus (N/m²) was calculated from the following equation where gradient (N/m) was calculated by the ratio of force at 2% and 3% strain:

$$Young ’ smodulus ( N / {m}^{2} ) = \frac{Gradient ( N / m ) \times height of the gel ( m )}{Area of the gel ( {m}^{2} )}$$

Area of the gel was the contact area of the gel with the probe which was calculated from the surface area of the uniform cylinder molds.

The texture profile analysis (TPA) was carried out with 20%, 30% and 75% strain to mimic different components of mastication. Pre-test, test, and post-test speeds were 1 mm/s, and the trigger force was 5 g. Strain height was measured automatically during compression. Hardness, adhesiveness, resilience, cohesiveness, springiness, and chewiness parameters were calculated from the TPA data which were analyzed with the Exponent connect software. Gumminess parameter was excluded from the analysis since it is comparable to chewiness. Gumminess is valid for semi-solid materials, whereas chewiness is applicable for solids (Bourne, 2002).

Syneresis measurements. Syneresis measurements were based on weight loss of the gels upon freezing at −30° C. and thawing at ambient temperature (approximately 22° C.). The gel was weighed and sealed with an airtight foil, i.e., aluminum blister. After freezing for 12 h and thawing for 4 h at ambient temperature, the gel was weighed again and the difference in gel weight was normalized to percentage loss.

Water activity measurements. The water activity (a_{w}) of the gels was measured with HygroPalm HC2-AW (Rotronic, Switzerland) at ambient temperature. The sample was placed into the measurement chamber and the water activity was recorded after 15 min.

Droplet size analyses. Droplet sizes of the emulsion gels were measured with the Mastersizer 3000 Hydro MV (Malvern, UK). After AEG and PEG were prepared, approximately 2 g of sample was dissolved in water at 50° C. For GEG, the solvent was 0.1 M HCl. Solutions were added to the water cell drop by drop until an obscuration rate of 5-18% was obtained. The dispersant refractive index was set to 1.330 and 1.470, for water and corn oil, respectively. Particle absorption index was 0.010 for all emulsion gels. The data collected from the detectors was analyzed by the Mastersizer software. The software provided average droplet size parameters (D [4, 3] volume mean diameter and D [3, 2] surface mean diameter) of five measurements, as well as the droplet size distribution of each emulsion gel.

In vitro lipolysis. In vitro lipolysis was performed either as gastrointestinal (gastric lipolysis followed by intestinal lipolysis) stage or intestinal stage only. Digestion in the gastric stage was not monitored. Therefore, the gastric stage should be considered as a pretreatment before the intestinal stage. In addition to emulsion gels (GEG, PEG, and AEG), control gels of each emulsion gel (GC, PC and AC) were tested with gastrointestinal in vitro lipolysis setup. Control gels were prepared as described above for the emulsion gels apart from not containing oil or emulsifier (Table 9). To mimic mastication, the polysaccharide gels were pushed through a metal mesh with pore size 1.7 mm (Endecott, England), whereas gelatin gels were dissected with a scalpel into similar size pieces as the polysaccharide gels. The in vitro lipolysis protocol previously reported by Dille and Draget, (2021) was followed with modifications.

TABLE 9
Compositions of control gels for in vivo lipolysis.
All ingredients are given as weight percentage (w/w).

| | Agar emulsion | Pectin emulsion | Gelatin emulsion |
|---|---|---|---|
| Ingredient | gel control (AC) | gel control (PC) | gel control (GC) |
| Water | 52.60 | 50.71 | 46.00 |
| Agar | 2.00 | — | — |
| Pectin | — | 2.80 | — |
| Gelatin | — | — | 8.60 |
| Xylitol | 28.40 | 28.40 | 28.40 |
| Sorbitol | 14.00 | 14.00 | 14.00 |
| MA | 1.00 | 1.50 | 1.00 |
| TCD | 2.00 | 2.00 | 2.00 |
| Ascorbic Acid | — | 0.35 | — |
| CCT | — | 0.24 | — |

In vitro lipolysis of the gels was performed at 37° C. For the gastric stage, 1.5 g of gel was mixed with a NaCl solution. Rabbit gastric extract was added, and the pH was quickly and manually reduced to 3. The pH combination electrode was A 1622 M DI (SI Analytics, Germany) coupled to the titrator 7000-M1/20, TitroLine (SI Analytics, Germany). The simulated gastric fluid (SGF) was kept at constant stirring for 1 h. The final volume of the SGF was 10 ml, and the concentrations of NaCl and gastric lipase were 80 mM and 4 mg/ml, respectively. Afterwards, the contents of the SGF were transferred to a beaker containing simulated intestinal fluid (SIF).

The SIF was formed by adding 450 mg bile extract porcine (Sigma, USA, lot #SLCC9272) and water into the beaker and stirring the contents until the bile extract was fully dissolved. 12.5 ml salt mix solution (40 mM CaCl₂)/536 mM NaCl/dH₂O) was added to the beaker and kept in magnetic stirring for 10 min. The pH was adjusted to 7 by manually adding droplets of 1 mM and 0.1 mM NaOH. Pancreatin from porcine pancreas (Sigma, USA, lot #SLBZ5739) and lipase from porcine pancreas (Sigma, USA, lot #SLBZ7254) were dissolved in water in separate Eppendorf tubes through vortex mixing. The titrator was set to maintain the pH at 7 by adding droplets of 0.1 mM NaOH. The pH-stat method was initiated after simultaneously adding 1 ml of each enzyme mixture into the beaker. The final concentrations of the SIF components were 9 mg/ml bile extract porcine, 10 mM CaCl₂), 134 mM NaCl, 1.2 mg/ml pancreatin and 1.2 mg/ml lipase. The final volume of the SIF was 50 ml. The SIF was kept at constant stirring for 1 h while the data from the pH-stat method were recorded through the titrator.

For tests with only the intestinal in vitro lipolysis, the salt mix contained 40 mM CaCl₂) and 600 mM NaCl to reach a final concentration of 10 mM CaCl₂) and 134 mM NaCl in SIF without the SGF. For the in vitro lipolysis curves, the total lipolysis was assumed to start when pH initially reached pH 7, i.e., the data points until pH 7 were removed and the total NaOH consumption was calculated after this point. The initial rate of the in vitro lipolysis reaction was calculated from the slope of the total lipolysis curve between 4 and 80 s.

Microscopy. Photomicrographs of the emulsion gels were taken after 1 h of gastric lipolysis using inverted light microscope Axio Observer Z1 (Carl Zeiss Microscopy GmbH, Germany). A small amount of SGF was transferred with a Pasteur pipette on the microscope slide, covered with a cover slip and examined using the microscope. The ZEISS ZEN pro digital imaging software (Version 2.3) was used to photograph the samples.

Statistical analyses. All statistical analyses were performed using IBM® SPSS® Statistics software version 28.0.1.0 (142). One-way analysis of variance (ANOVA) with post-hoc Tukey's honest significant difference (HSD) test was performed. Statistically significant differences were reported if p<0.05.

### Results and Discussion

The emulsion gels were characterized and compared using water activity (a_{w}), syneresis, SAOS, large scale deformation, and droplet size measurements. To study the impact of oil in the polysaccharide gels, AEG and PEG were also compared to their “without oil” (WO) versions, AWO and PWO, respectively. In addition, AWO and AEG were compared to a pure agar gel (AG). In the WO gels, the aqueous phase was identical to the emulsion gels; for any ingredient in the WO gel, solute/water ratio is equal to its corresponding emulsion gel. Because of this, similar a_{w} was observed for emulsion gels and their WO versions. The a_{w} of different emulsion gels were proportional to the water they contain in the formulation (Table 8, Table 10). The a_{w} of AG was much higher than AWO and AEG, highlighting the importance of co-solutes to decrease the a w of food systems below 0.85 to prevent microbial growth as recommended by the U.S. Food and Drug Administration (2018).

TABLE 10
Water activity (a_{w}) and syneresis of the gels presented
as % water loss. For a_{w} and syneresis measurements, three
and 6-9 replicates per each sample were analyzed, respectively.
For a_{w}, the standard deviation was smaller than 0.01 for each gel. The
abbreviations used for the gels are given in Table 8. * This result was due
to minor adhesion of the gel to the aluminum blister. Different letters in
superscript indicate statistically significant differences between gels.
For both a_{w} and syneresis p < 0.001 between different gels.

| Gel | a_{w} | Average syneresis (%) |
|---|---|---|
| AG | 0.94 ^{d} | 5.6 ± 0.7 ^{d} |
| AWO | 0.77 ^{bc} | 4.7 ± 0.6 ^{c} |
| AEG | 0.77 ^{c} | 3.2 ± 0.2 ^{b} |
| PWO | 0.74 ^{a} | 1.5 ± 0.3 ^{a} |
| PEG | 0.76 ^{b} | 1.7 ± 0.4 ^{a} |
| GEG | 0.73 ^{a} | 1.0 ± 0.5 * |

Syneresis, i.e., expulsion of water from a gel, is a common phenomenon for agar gels (Armisen & Gaiatas, 2009). AG showed the highest syneresis (Table 10). The addition of sugar alcohols to AG significantly lowered syneresis for AWO, in accordance with lower a_{w}. The inclusion of oil in the formulation further reduced syneresis for AEG significantly. In a previous study with alginate emulsion gels, this was suggested to be due to oil droplets acting as barriers for water transport (Levi ć et al., 2015; Lin et al., 2020a). It is known that increasing agar concentrations result in lower syneresis (Banerjee & Bhattacharya, 2011). The current data suggest that the inclusion of sugar alcohols and oil also contribute to reducing syneresis due to a reduced amount of water as well as a greatly reduced a_{w} (free water) in the system. Pectin gels are known to exhibit syneresis during storage as a result of aging (Rao, Van Buren & Cooley, 1993). Although syneresis was lower for both PEG and PWO compared to agar gels, the inclusion of oil did not reduce the syneresis of the pectin gel (Table 10).

GEG was not included in the statistical tests for syneresis (Table 10). In this system, syneresis was considered negligible since no sweating was observed. This is in accordance with literature (Mizrahi, 2010). In general, syneresis in food gels is an undesirable phenomenon since it may impact the quality and stability of the product (Banerjee & Bhattacharya, 2011; Mizrahi, 2010). However, for adhesive gels which exhibit stickiness to their packaging material, a controlled amount of syneresis may promote lubrication due to surface liquid (Saha & Bhattacharya, 2010). During the syneresis measurements no stickiness was observed with agar or pectin gels.

Small amplitude oscillatory shear measurements. Although agar/water ratio was kept constant for the agar gels (AG, AWO, and AEG), it should be noted that AG had a much higher overall concentration of agar in the total formulation. In addition, water-soluble polyols increase the total volume of the water phase, resulting in a lower agar/water phase ratio for AWO and AEG. Consequently, AG had the significantly highest G′ₘₐₓ among agar gels, followed by AWO and AEG, although without significant differences (Table 11). In accordance with the literature, inactive fillers decrease gel strength compared to an oil-free gel (Dickinson & Chen, 1999; Dille, Haug & Draget, 2021b). Similarly, PWO had a higher G′ₘₐₓ than PEG, although the difference was not statistically significant. Among emulsion gels, AEG had the highest and PEG had the lowest G′ₘₐₓ, respectively.

TABLE 11
G′ₘₐₓ, Tₘ, and T_{g} of emulsion gels, their “without oil”
versions and a pure agar gel. The results are presented as the average
of three independent replicates ± standard deviation. The abbreviations
used for the gels are given in Table 8. N/A indicates that data
could not be obtained since the phase angle has not reached 45°
with the current temperature gradient. The results were obtained
with a temperature gradient of 2° C./min and are expected to
vary between different temperature gradients. Different letters
in superscript indicate statistically significant differences between
gels. For all three parameters p < 0.001 between different gels.

| | G′ₘₐₓ (kPa) | Tₘ ° C. | T_{g} ° C. |
|---|---|---|---|
| AG | 145.9 ± 33.0 ^{d} | N/A | 50.7 ± 6.8 ^{b} |
| AWO | 73.9 ± 1.7 ^{c} | 87.4 ± 1.1 ^{c} | 39.1 ± 1.1 ^{a} |
| AEG | 44.1 ± 7.8 ^{bc} | 90.1 ± 0.2 ^{c} | 40.2 ± 1.3 ^{a} |
| PWO | 6.1 ± 0.2 ^{ab} | 50.5 ± 2.2 ^{b} | 36.3 ± 1.0 ^{a} |
| PEG | 3.2 ± 0.3 ^{a} | 54.2 ± 2.7 ^{b} | 38.3 ± 1.8 ^{a} |
| GEG | 16.8 ± 1.7 ^{ab} | 45.3 ± 1.3 ^{a} | 37.0 ± 0.5 ^{a} |

Although the Tₘ of AG could not be obtained, it is expected to be above 90° C., due to high agar concentration (Lahrech, Safouane & Peyrellasse, 2005). The Tₘ and T_{g} of AWO and AEG were similar with a slightly higher Tₘ for AEG. The Tₘ of AEG (90° C.) was significantly higher than PEG (54° C.) and GEG (45° C.) which were also significantly different (Table 11). A high Tₘ may be advantageous for the storage and textural stability of polysaccharide-based gel products in warm climates. The stability of gelatin gels is known to be compromised at high storage temperatures due to the acid hydrolysis of gelatin (Baydin et al., 2022; Van den Bosch & Gielens, 2003). However, the high Tₘ of AEG may present challenges during commercial production since it would require critical temperature control to ensure the quality of the final product. Although agar gels have a reversible gelation process without significant compromising the gelling properties (Imeson, 2009), AEG had a lower G′ₘₐₓ after re-melting the gel (data not shown). For industrial production, PEG may be a more manufacturable formulation alternative to GEG since it has relatively similar Tₘ and T_{g} values to GEG and it did not exhibit large decreases in G′ₘₐₓ after re-melting (data not shown). It should be noted that PEG may pose challenges for industrial applications due to the complexity and sensitivity of its formulation to, e.g., pH and calcium concentration (Burey, Bhandari, Rutgers, Halley & Torley, 2009; Zeeb et al., 2021).

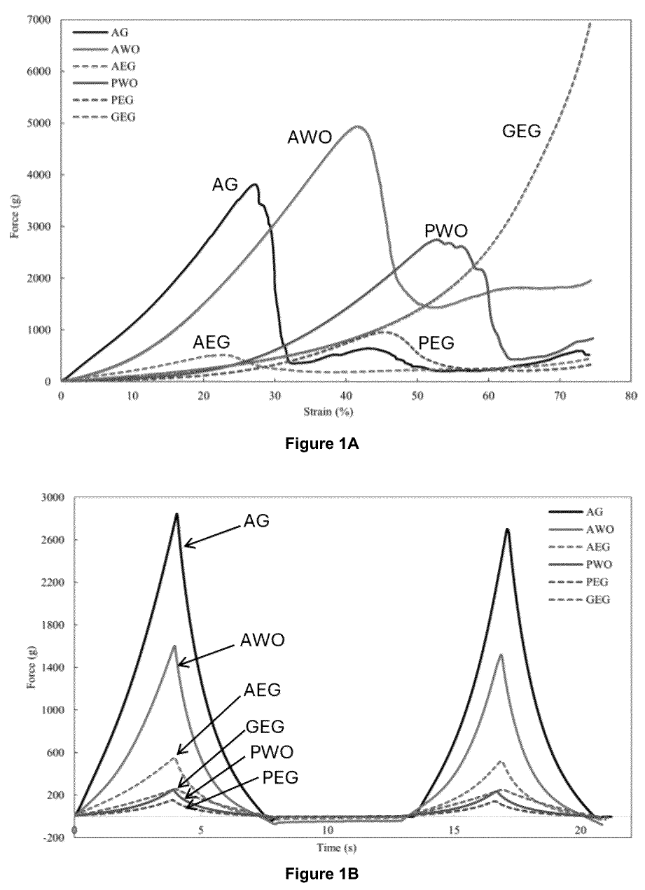

Texture characteristics of the gels. Consumer acceptance of emulsion gels for oral delivery highly depend on their mechanical properties, which describe their response to deformation, such as mastication, i.e., chewing (Aguayo-Mendoza et al., 2020). Texture characteristics of the gels were analyzed with a single compression and TPA tests with three maximum strains. The average curves of single compression with 75% strain are shown in FIG. 1A. The parameters of this test demonstrated that AG had the significantly highest Young's modulus among all gels, indicating high stiffness/rigidity. Although the inclusion of sugar alcohols (AWO) significantly decreased the Young's modulus, it significantly increased the maximum force and strain at failure of the agar gel (Table 12). The lower Young's modulus of AWO is due to a lower overall agar concentration in the formulation compared to AG. Strain at failure coincides with the sensory deformability of the material (Koç et al., 2019). As discussed above, AG had a much higher overall concentration of agar in the total formulation compared to AWO and AEG. The stiffness order of agar gels (AG>AWO>AEG) obtained with single compression were in accordance with the G′ₘₐₓ values obtained by SAOS measurements (Table 12).

TABLE 12
Parameters of large scale deformation (75% strain single compression)
of the gels with texture analyzer. The results are given as the average
of 6-9 replicates (obtained from a single batch) ± standard deviation.
The abbreviations of the gels are given in Table 8. Different letters
in superscript indicate statistically significant differences between
gels. For all three parameters p < 0.001 between different gels.

| | Young's modulus (kN/m²) | Max force (g) | Strain at failure (%) |
|---|---|---|---|
| AG | 552.4 ± 31.7 ^{e} | 3439 ± 906 ^{b} | 25 ± 5 ^{a} |
| AWO | 182.0 ± 24.7 ^{d} | 4993 ± 227 ^{c} | 42 ± 1 ^{b} |
| AEG | 95.0 ± 1.9 ^{c} | 523 ± 39 ^{a} | 22 ± 1 ^{a} |
| PWO | 21.5 ± 1.1 ^{ab} | 3299 ± 573 ^{b} | 56 ± 4 ^{c} |
| PEG | 12.6 ± 0.3 ^{a} | 975 ± 100 ^{a} | 46 ± 1 ^{b} |
| GEG | 46.2 ± 3.7 ^{b} | 7349 ± 150 ^{d} | >75 ^{d} |

Both agar and pectin WO gels had significantly higher Young's modulus, maximum force and strain at failure, compared to their corresponding emulsion gels, although the difference was not significant for the Young's modulus of PWO and PEG (Table 12). The inclusion of oil in the agar and pectin formulations resulted in a more brittle and weaker texture as the oil droplets behave as inactive fillers (Kim et al., 1997, 1999; Zhang et al., 2022). The emulsified oil droplets may provide an inactive filler effect with the continuous phase which results in a weaker polysaccharide gel network and provide possible failure zones for crack propagation (Dickinson, 2012; Dille, Draget & Hattrem, 2015; Sala, van de Velde, Stuart & van Aken, 2007). AEG showed significantly higher brittleness than PEG (Table 12).

GEG had the significantly highest maximum force among all gels, and it did not fail at 75% strain, pointing to the ductile texture of the gel (FIG. 1A, Table 12). The polysaccharide emulsion gels had a significantly lower maximum force, and they were significantly more brittle than GEG (Table 12). The brittleness of polysaccharide gels has been attributed to their high chain stiffness, as opposed to gelatin with long and flexible cross-links (Van Vliet & Walstra, 2017). Compared to AEG, although significantly different, PEG had strain at failure and maximum force values closer to GEG, suggesting a more similar sensory perception. Young's modulus and strain at failure parameters were significantly different between the emulsion gels (p<0.001), pointing to their different deformation characteristics.

Gelatin gels have a unique chewy texture that is familiar to consumers of gelatin-based jelly desserts, gummies, and confectionery (Schrieber & Gareis, 2007). This texture is difficult to be mimicked with polysaccharides since polysaccharide gels typically do not have a similar elastic/chewy texture (Haug, Draget & Smidsrød, 2004; Karim & Bhat, 2008). The texture of the gels was analyzed with standard TPA tests at three different strains (20%, 30%, and 75%) which represent a variety of strain at failure values obtained from the single compression test. These strain values also represent different anatomical components of mastication such as the tongue, hard palate and teeth (Arai & Yamada, 1993). It has been shown that different degrees of compression, i.e., maximum strain, impact TPA parameters (Bourne & Comstock, 1981). Standard TPA parameters (hardness, adhesiveness, cohesiveness, resilience, springiness, and gumminess) were obtained for each gel at three different strains.

FIG. 1B shows TPA curves of the gels with 20% strain, which is below the strain at failure for all gels. The textural properties of the gels are shown in Table 13. Similar to the Young's modulus values, hardness of the gels was in the following order for agar: AG>AWO>AEG, and PWO>PEG for pectin gels with significant differences, pointing out to weaker gel structure due to the inactive filler effect (Farjami & Madadlou, 2019; Koç et al., 2019). Hardness, gumminess, and adhesiveness of the emulsion gels were in the following order: AEG>GEG>PEG, and AWO showed the significantly highest adhesiveness among all gels. Resilience, springiness, and cohesiveness parameters describe the structural integrity of the gel after the first compression. The springiness value was close to 100, and cohesiveness value was one for all gels since none of the gels fractured at this strain. The significantly lower resilience of the polysaccharide emulsion gels, compared to gelatin emulsion gel, indicated some structural damage, especially for AEG which had a strain at failure closer to 20% strain than PEG (Table 13). All parameters of the 20% strain TPA test (excluding cohesiveness) were significantly different between the gels (p<0.001).

TABLE 13
Textural properties of the gels obtained with 20% strain TPA test. The results
are given as the average of (7-10) gels (obtained from a single batch) ±
standard deviation. The abbreviations and compositions of the gels are given
in Table 8. Cohesiveness value for all the gels was 1 ± 0. Different letters
in superscript indicate statistically significant differences between gels.

| | Hardness | Adhesiveness | Resilience | Springiness | |
|---|---|---|---|---|---|
| Gel | (g) | (g · sec) | (%) | (%) | Gumminess |
| AG | 2853 ± 175 ^{e} | −30 ± 24 ^{c} | 65 ± 1 ^{c} | 91 ± 1 ^{a} | 2404 ± 160 ^{e} |
| AWO | 1614 ± 53 ^{d} | −271 ± 83 ^{a} | 61 ± 1 ^{b} | 93 ± 1 ^{b} | 1366 ± 50 ^{d} |
| AEG | 558 ± 25 ^{c} | −101 ± 16 ^{b} | 56 ± 1 ^{a} | 96 ± 1 ^{c} | 459 ± 20 ^{c} |
| PWO | 251 ± 4 ^{b} | −9 ± 4 ^{c} | 78 ± 1 ^{e} | 99 ± 1 ^{d} | 227 ± 4 ^{b} |
| PEG | 155 ± 4 ^{a} | 4 ± 2 ^{c} | 76 ± 1 ^{d} | 100 ± 0 ^{d} | 139 ± 4 ^{a} |
| GEG | 255 ± 11 ^{b} | −14 ± 7 ^{c} | 92 ± 2 ^{f} | 99 ± 1 ^{d} | 249 ± 11 ^{b} |

The only fractured gel during 30% strain TPA test was AEG, and therefore, its cohesiveness value was zero unlike the other gels which had a cohesiveness value of one (Table 14). Consequently, it also had significantly lower resilience and springiness than the other gels (p<0.001). Compared to 20% strain TPA test, the springiness and resilience of all gels decreased, pointing to structural damage. Similar to the 20% strain TPA test, polysaccharide emulsion gels had significantly lower hardness and gumminess compared to their WO gels, indicating the negative impact of the inactive filler effect on these parameters. The hardness and adhesiveness of the emulsion gels followed the same order as 20% strain TPA (Table 13, Table 14). The 30% strain TPA curves of the gels are shown in FIG. 5. All parameters of the 30% strain TPA test (excluding cohesiveness) were significantly different between the gels (p<0.001).

TABLE 14
Textural properties of the gels obtained with 30% strain TPA test. The results are
given as the average of (6-10) gels (obtained from a single batch) ± standard
deviation. The abbreviations and compositions of the gels are given in Table 8.
Cohesiveness value for all the gels was 1 ± 0 except for AEG (0 ± 0).
Different letters in superscript indicate statistically significant differences between gels.

| | Hardness | Adhesiveness | Resilience | Springiness | |
|---|---|---|---|---|---|
| | (g) | (g · sec) | (%) | (%) | Gumminess |
| AG | 5379 ± 118 ^{e} | −25 ± 10 ^{b} | 50 ± 0 ^{c} | 88 ± 0 ^{ab} | 3963 ± 138 ^{f} |
| AWO | 3190 ± 86 ^{d} | −365 ± 149 ^{a} | 46 ± 1 ^{b} | 92 ± 2 ^{bc} | 2370 ± 56 ^{e} |
| AEG | 524 ± 52 ^{b} | −93 ± 35 ^{b} | 14 ± 2 ^{a} | 85 ± 7 ^{a} | 159 ± 32 ^{a} |
| PWO | 816 ± 16 ^{c} | −46 ± 7 ^{b} | 55 ± 1 ^{d} | 95 ± 1 ^{cd} | 646 ± 8 ^{d} |
| PEG | 427 ± 9 ^{a} | −13 ± 4 ^{b} | 56 ± 1 ^{d} | 98 ± 1 ^{d} | 341 ± 5 ^{b} |
| GEG | 490 ± 23 ^{ab} | −35 ± 14 ^{b} | 87 ± 1 ^{e} | 98 ± 1 ^{d} | 479 ± 21 ^{c} |

All polysaccharide gels fractured at 75% strain TPA test (reflected by the cohesiveness values). At this strain, GEG had a significantly higher hardness and gumminess than the polysaccharide emulsion gels (FIG. 6). Although it did not fracture at 75% strain, GEG's low resilience indicated structural damage. The hardness of GEG increased with increasing maximum strain of the TPA (Table 13, Table 14, Table 15). At 75% strain, the gel is close to its elastic limit, resisting deformation to a larger extent (Mazumder, Roopa & Bhattacharya, 2007). The presence of oil significantly decreased the hardness and gumminess of the polysaccharide gels, whereas it had little effect on the resilience, cohesiveness, and springiness. Although not significantly different, PEG had a slightly higher hardness than AEG. All parameters of the 75% strain TPA test (excluding cohesiveness) showed statistically significant differences between the gels (p<0.001).

TABLE 15
Textural properties of the gels obtained with 75% strain TPA test. The results are
given as the average of (8-13) gels (obtained from a single batch) ± standard
deviation. The abbreviations and compositions of the gels are given in Table 8.
Cohesiveness value for all the gels was 0 ± 0 except for GEG (1 ± 0).
Different letters in superscript indicate statistically significant differences between gels.

| | Hardness | Adhesiveness | Resilience | Springiness | |
|---|---|---|---|---|---|
| | (g) | (g · sec) | (%) | (%) | Gumminess |
| AG | 6946 ± 610 ^{c} | −6 ± 6 ^{b} | 1 ± 1 ^{a} | 38 ± 8 ^{a} | 159 ± 86 ^{a} |
| AWO | 7839 ± 583 ^{d} | −20 ± 31 ^{b} | 1 ± 1 ^{a} | 40 ± 11 ^{a} | 347 ± 136 ^{b} |
| AEG | 1320 ± 61 ^{a} | −92 ± 39 ^{b} | 3 ± 1 ^{b} | 52 ± 20 ^{ab} | 114 ± 28 ^{a} |
| PWO | 5006 ± 398 ^{b} | −82 ± 47 ^{ab} | 1 ± 0 ^{a} | 58 ± 12 ^{b} | 327 ± 48 ^{b} |
| PEG | 1494 ± 86 ^{a} | −13 ± 18 ^{ab} | 2 ± 0 ^{b} | 49 ± 12 ^{ab} | 104 ± 15 ^{a} |
| GEG | 7430 ± 244 ^{cd} | −159 ± 130 ^{a} | 50 ± 0 ^{c} | 89 ± 2 ^{c} | 5842 ± 181 ^{c} |

Both single compression and 75% strain TPA test compressed the gels to the same extent. However, maximum force and fracture strain results of the single compression test (0.1 mm/s) were lower than those obtained from the hardness results of the TPA test (1 mm/s). This may be due to the compression speed differences of the two tests. A slower compression rate gives the gel more time to relax and dissipate the applied force through friction between structural components of the gel at higher deformation speeds (Pons & Fiszman, 1996; Sala, Van Vliet, Stuart, Van Aken & Van de Velde, 2009). In previous studies, compression rates of 2-3 mm/s were suggested to be more physiologically relevant to mastication (Pons & Fiszman, 1996; Rosenthal, 2010).

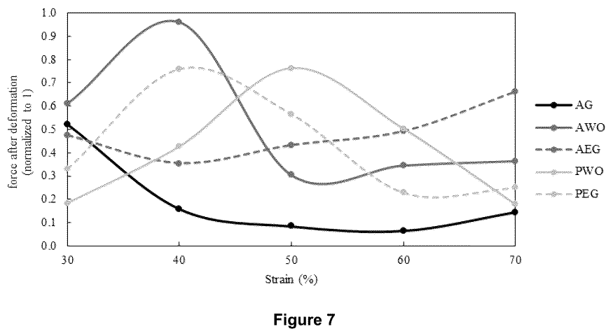

The texture analyses indicated that at high strains (75%), the addition of sugar alcohols to the pure agar gel resulted in higher hardness, whereas lower strains (20 and 30%) showed the opposite. The addition of sugar alcohols resulted in a less brittle texture and further addition of oil to the agar formulation led to a lower hardness at all strains. Structure maintained in the gel after deformation was as followed: AEG>AWO>AG, suggesting that the addition of polyols and oil to a pure agar gel resulted in a texture with higher structural integrity (FIG. 7). AEG had a harder, firmer, and more brittle texture than GEG. The addition of oil weakened the pectin gels at all strains. PEG had lower hardness than both AEG and GEG. However, it was less brittle and had higher resilience, cohesiveness, and springiness than AEG, potentially making its mouthfeel more similar to GEG.

Texture analyses demonstrated different characteristics of agar, pectin, and gelatin emulsion gels. These differences will impact the sensory perception, aroma release profile, and dissolution time of the emulsion filled gels (Sala et al., 2008). A lower pH of PEG (pH 4) may result in faster flavor release than AEG or GEG (pH 4.5) (Hansson, Andersson, Leufven & Pehrson, 2001). One of the most commonly used biopolymers in vegan confectionery is pectin, which may have a familiar texture for vegan consumers (Seremet et al., 2020). In Asia, AEG may have higher consumer acceptance since agar has traditionally been used as a food ingredient for centuries (Sousa et al., 2021).

Droplet size and in vitro lipolysis of emulsion gels. The droplet size of an emulsion is known to influence the digestion, uptake, and bioavailability of TAGs (Dille, Baydin, Kristiansen & Draget, 2021a). Especially the surface mean diameter, i.e., D [3, 2], is important since a lower average provides a larger surface area accessible for the digestive enzymes. For both D [3, 2] and D [4, 3], GEG had the smallest droplet size, whereas PEG had the largest (Table 16). Both droplet size averages were significantly different between emulsion gels (p<0.001).

TABLE 16
The D[4 , 3] and D[3 , 2] droplet size averages
of agar (AEG), pectin (PEG) and gelatin (GEG) emulsion gels.
The D[4 , 3] and D[3 , 2] droplet size averages
are given as the average of three replicate gels with five
measurements ± standard deviation. The results of the
in vitro lipolysis are given as the average of two experiments
± standard deviation. Different letters in superscript
indicate statistically significant differences between groups.

| | | | Initial lipolysis | |
|---|---|---|---|---|
| | Droplet size averages | | rate (μmol FFA/sec) | |
| | D[4, 3] (μm) | D[3 , 2] (μm) | Gastrointestinal | Intestinal |
| AEG | 13.2 ± 1.5 ^{a} | 3.0 ± 0.8 ^{a} | 2.75 ± 0.07 ^{a} | 2.45 ± 0.07 ^{a} |
| PEG | 31.5 ± 5.1 ^{b} | 9.7 ± 1.7 ^{b} | 4.30 ± 0.14 ^{a} | 3.50 ± 1.13 ^{a} |
| GEG | 1.0 ± 0.2 ^{c} | 0.6 ± 0.0 ^{c} | 24.85 ± 7.57 ^{b} | 35.15 ± 6.29 ^{b} |

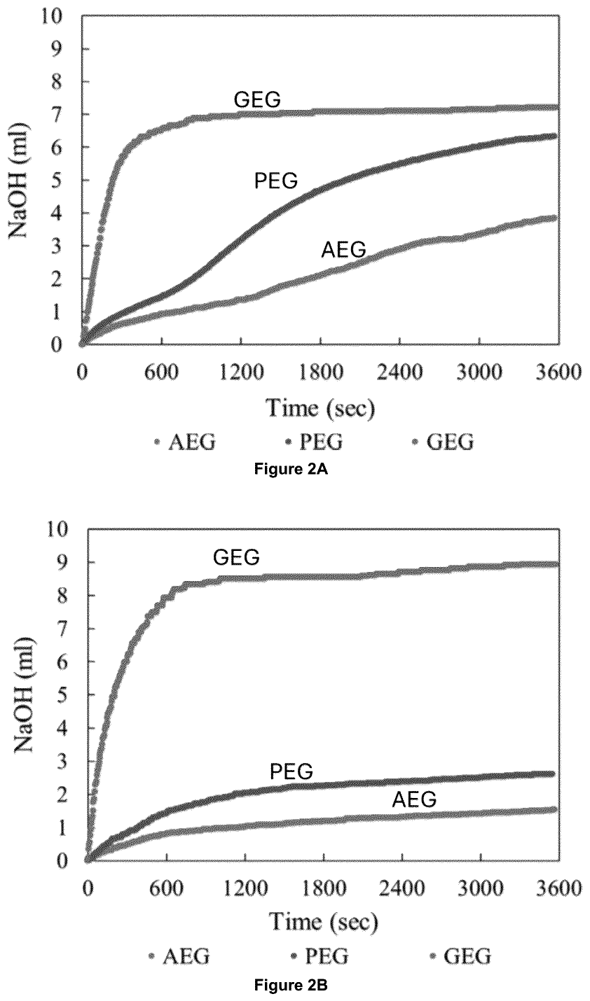

The lipolysis of the emulsion gels was studied through in vitro experiments. To evaluate the decrease in pH due to the experimental setup, gastrointestinal lipolysis was performed with a “blank” sample (water instead of a gel), as a background control. The total NaOH consumption was 0.40±0.11 ml (n=3). This indicates the background lipolysis reaction occurring, possibly due to the presence of lipid impurities in the bile extract or the lipolysis of pancreatin (Larsen, Sassene & Müllertz, 2011). During gastrointestinal lipolysis, the net NaOH consumption is calculated as the total consumption of the control gel (AC, PC, and GC) subtracted from the total consumption of the corresponding emulsion gel (AEG, PEG, and GEG) (see above). The control gels were only subjected to gastrointestinal lipolysis, i.e., control gels have not been tested in the intestinal stage. In the gastrointestinal stage, GEG had the highest net consumption with 6.09±0.44 ml, followed by PEG with 5.22±0.44 ml and lastly AEG with 3.18±0.34 ml (FIG. 2A). The total NaOH consumption in the intestinal stage was 8.93±0.67 ml for GEG, followed by 2.61±0.13 ml for PEG and 1.53±0.01 ml for AEG (FIG. 2B). The reason for GEG having a higher total NaOH consumption in the intestinal stage compared to the gastrointestinal stage may be because of the free fatty acids released from the triacylglycerols (TAGs) in the SGF becoming deprotonated as the pH is raised to 7 before the intestinal stage. Therefore, less TAGs would be available in the intestinal stage to be hydrolyzed and release fatty acids that are detected by the pH-stat method since some of the TAGs were already hydrolyzed to diacylglycerols or monoacylglycerols in the gastric stage. The lipolysis curves of the gels are shown in FIG. 2A-FIG. 2B.

In both gastrointestinal lipolysis and intestinal lipolysis, the titration curves of polysaccharide emulsion gels have not reached a plateau during 1 hour of titration. A higher total NaOH consumption might have been recorded for the gels if the intestinal titration was prolonged. A longer duration of titration could, however, have given more physiologically relevant results since the gastrointestinal transit time in humans is longer than 1 hour (Read, Al-Janabi, Holgate, Barber & Edwards, 1986; Worsøe et al., 2011).

The initial lipolysis rate of the emulsion gels followed the same order for both gastrointestinal and intestinal lipolysis: GEG>PEG>AEG. In both gastrointestinal (p<0.025) and intestinal (p<0.005) lipolysis, the initial rate of different emulsion gels was significantly different with GEG being different from the polysaccharide emulsion gels (Table 16). Larger oil droplets are lipolyzed at a slower rate compared to small oil droplets which provide a larger surface area for the digestive enzymes to adsorb to (Dille et al., 2021a). Although AEG had a smaller droplet size than PEG, its initial lipolysis rate was slower. The initial rate of AEG and PEG were lower in intestinal stage, compared to their relative rate in the gastrointestinal stage. However, the opposite was observed for GEG, with 1.4× faster initial lipolysis in the intestinal stage.

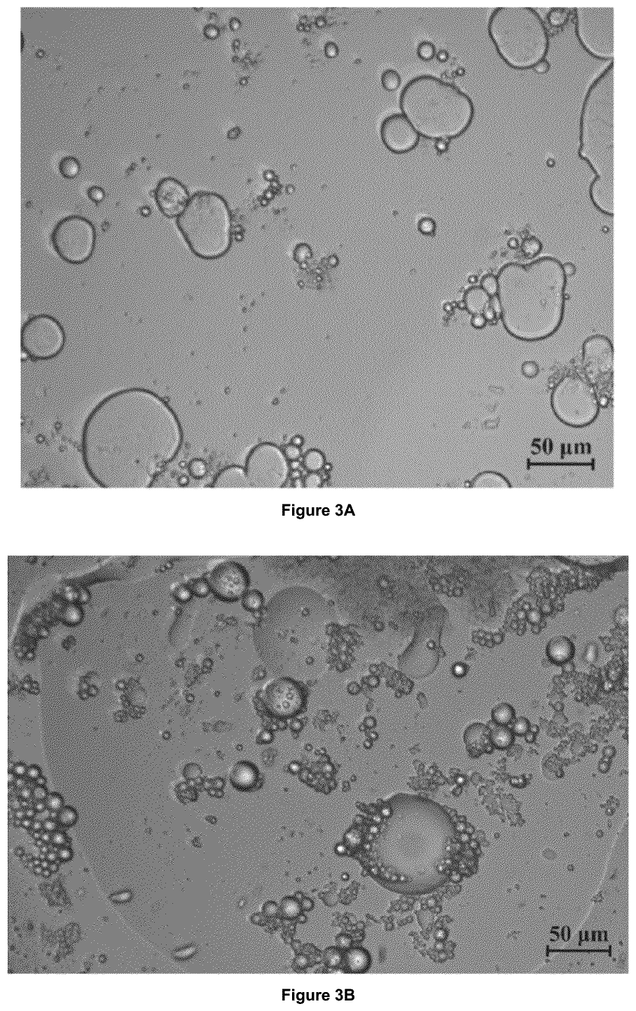

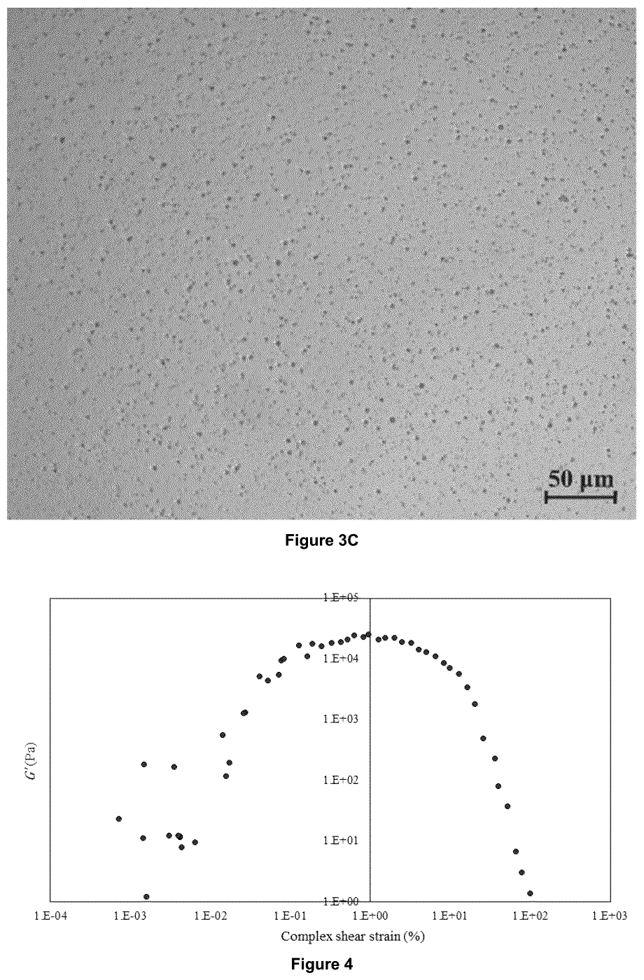

The photomicrographs of the emulsion gels after the gastric stage showed coalescence of oil droplets in AEG (FIG. 3A). Similar observations have been reported for citrem stabilized emulsions in the gastric stage at pH 1.3 and 3, and this was suggested to be due to the lowered emulsifying capacity of citrem with the loss of negative charges at lower pH values (Lamothe, Desroches & Britten, 2019, 2020; Sørensen et al., 2008). The photomicrographs of PEG indicated flocculated oil droplets (FIG. 3B). At low pH values, emulsions stabilized by soybean lecithin have been shown to destabilize due to reduced emulsifying capacity of the emulsifier (Comas, Wagner & Tomás, 2006; Lin & Wright, 2018; Lin, Wang, Li & Wright, 2014). Simultaneously, pectin may contribute to the stabilization of the emulsion, keeping the emulsion droplets in an aggregated but not coalesced state, because of bridging flocculation (Ngouémazong, Christiaens, Shpigelman, Van Loey & Hendrickx, 2015).

Coalescence and flocculation of oil droplets reduce the available substrate area for digestive enzyme adsorption and reduce the total extent and rate of lipolysis (Li et al., 2011; McClements, 2018). The lipolysis curves of gastrointestinal lipolysis of AEG and PEG show an increase at approximately 1000 s which was not observed for the intestinal lipolysis without a gastric pretreatment (FIG. 2A-FIG. 2B). For PEG, this increase in NaOH consumption may be due to the increase of available substrate for the lipases in the intestinal stage at pH 7 which may disperse the flocs and create more available surface. It should also be noted that calcium ions are absent in the gastric stage. Calcium helps with the precipitation of FFAs, removing them from the surface of the oil droplets, and increasing the accessibility of the surface to the digestive enzymes (Minekus et al., 2014). Furthermore, the absence of calcium in SGF may mediate dissolution of the pectin network since calcium binds to pectin and facilitates its gelling (Fraeye, Duvetter, Doungla, Van Loey & Hendrickx, 2010). For AEG, the increase may be due to the gel remaining in the lipolysis medium for a longer period (two hours in the gastrointestinal stage, instead of one hour in the intestinal stage), with coalesced oil droplets being lipolyzed further, exposing a larger substrate area for the lipases. This would also promote shrinking of oil droplets due to lipase activity, further resulting in droplet release from the gel network.

It should be noted that in vitro lipolysis does not directly indicate the outcome of lipolysis in vivo. More complex in vitro lipolysis experiments with greater physiological relevance have been developed (Brodkorb et al., 2019), and the limitations of in vitro lipolysis experiments are well known (Ghorbani & Abedinzade, 2013). The current simplified setup serves a means to compare the lipolysis potential of emulsion gels, prepared with different biopolymers which contain the same amount of oil. Lower initial lipolysis rate, as well as lower total NaOH consumption for AEG and PEG in both gastrointestinal and intestinal stages, compared to GEG, point to limitations for the digestibility of oils in polysaccharide emulsion gels. These limitations may suggest potential lower absorption of polysaccharide emulsion gels in vivo, and consequently result in lower bioavailability. However, in the human body, shear forces in the stomach may result in a higher extent of mechanical deformation of the polysaccharide gels, resulting in smaller gel particles exposing a larger surface area available to lipases.

### CONCLUSIONS

Emulsion gels using agar and pectin were prepared, characterized, and compared to a traditional gelatin emulsion gel. The higher melting temperature of agar emulsion gel may be advantageous in warm climates for enhanced stability of the final product, while it may pose difficulties in processability. The pectin emulsion gel had a more similar melting/gelling temperature to gelatin emulsion gel. The addition of sugar alcohols and oil to a pure agar gel decreased hardness and brittleness, potentially resulting in a more pleasant mouth-feel. Pectin emulsion gel had a softer texture than the agar emulsion gel, and a more similar texture to the gelatin emulsion gel. In the future, the textural properties of the emulsion gels should be further studied through a sensory panel using e.g., quantitative descriptive analysis. In vitro lipolysis studies showed a lower extent of lipolysis and slower initial lipolysis rate with polysaccharide emulsion gels compared to the gelatin emulsion gel. Absorption and bioavailability of these three systems should be further studied using an in vivo model, and preferably in humans. The polysaccharide emulsion gels can also be used for other applications, as food gels or for the oral delivery of pharmaceuticals.

### REFERENCES

Adepu, S., & Ramakrishna, S. (2021). Controlled drug delivery systems: Current status and future directions. Molecules (Basel, Switzerland), 26 (19), 5905. https://doi.org/10.3390/molecules26195905.
Aguayo-Mendoza, M., Santagiuliana, M., Ong, X., Piqueras-Fiszman, B., Scholten, E., & Stieger, M. (2020). How addition of peach gel particles to yogurt affects oral behavior, sensory perception and liking of consumers differing in age. Food Research International, 134, Article 109213. https://doi.org/10.1016/j.foodres.2020.109213.
Alcorta, A., Porta, A., Tárrega, A., Alvarez, M. D., & Vaquero, M. P. (2021). Foods for plant-based diets: Challenges and innovations. Foods (Basel, Switzerland), 10 (2), 293. https://doi.org/10.3390/foods10020293.
Alipal, J., Pu'ad, N. M., Lee, T., Nayan, N., Sahari, N., Basri, H. et al. (2021). A review of gelatin: Properties, sources, process, applications, and commercialisation. Materials Today: Proceedings, 42, 240-250 10.1016/j.matpr.2020.12.922
Arai, E., & Yamada, Y. (1993). Effect of the texture of food on the masticatory process. Japanese Journal of Oral Biology, 35 (4), 312-322. https://doi.org/10.11501/3093560.
Araki, C. (1956). Structure of the agarose constituent of agar-agar. Bulletin of the Chemical Society of Japan, 29 (4), 543-544.
Armisen, R., & Gaiatas, F. (2009). Agar. In Handbook of Hydrocolloids (pp. 82-107). Elsevier.
Banerjee, S., & Bhattacharya, S. (2011). Compressive textural attributes, opacity and syneresis of gels prepared from gellan, agar and their mixtures. Journal of Food Engineering, 102 (3), 287-292. https://doi.org/10.1016/j.jfoodeng.2010.08.025.
Baydin, T., Aarstad, O. A., Dille, M. J., Hattrem, M. N., & Draget, K. I. (2022). Long-term storage stability of type A and type B gelatin gels: The effect of Bloom strength and co-solutes. Food Hydrocolloids, Article 107535. https://doi.org/10.1016/j.foodhyd.2022.107535.
Bourne, M. C. (2002). Food texture and viscosity: Concept and measurement. Elsevier.
Bourne, M. C., & Comstock, S. H. (1981). Effect of degree of compression on texture profile parameters. Journal of Texture Studies, 12 (2), 201-216. https://doi.org/10.1111/j.1745-4603.1981.tb01232.x.
Brodkorb, A., Egger, L., Alminger, M., Alvito, P., Assunção, R., Ballance, S., et al., (2019). INFOGEST static in vitro simulation of gastrointestinal food digestion. Nature Protocols, 14 (4), 991-1014. https://doi.org/10.1038/s41596-018-0119-1.
Burey, P., Bhandari, B., Rutgers, R., Halley, P., & Torley, P. (2009). Confectionery gels: A review on formulation, rheological and structural aspects. International Journal of Food Properties, 12 (1), 176-210. https://doi.org/10.1080/10942910802223404.
Chen, J., & Dickinson, E. (1999). Effect of surface character of filler particles on rheology of heat-set whey protein emulsion gels. Colloids and Surfaces B: Biointerfaces, 12 (3-6), 373-381. https://doi.org/10.1016/S0927-7765 (98) 00091-5.
Chen, L., Remondetto, G. E., & Subirade, M. (2006). Food protein-based materials as nutraceutical delivery systems. Trends in Food Science & Technology, 17 (5), 272-283. https://doi.org/10.1016/j.tifs.2005.12.011.
Comas, D., Wagner, J., & Tomás, M. (2006). Creaming stability of oil in water (O/W) emulsions: Influence of pH on soybean protein-lecithin interaction. Food Hydrocolloids, 20 (7), 990-996. https://doi.org/10.1016/j.foodhyd.2005.11.006.
Da Silva, J. L., & Rao, M. (2006). 11 pectins: Structure, functionality, and uses. Food Polysaccharides and Their Applications, 353.
DeFelice, S. L. (1995). The nutraceutical revolution: Its impact on food industry R&D. Trends in Food Science & Technology, 6 (2), 59-61. https://doi.org/10.1016/S0924-2244 (00) 88944-X.
Dickinson, E. (2009). Hydrocolloids as emulsifiers and emulsion stabilizers. Food Hydrocolloids, 23 (6), 1473-1482. https://doi.org/10.1016/j.foodhyd.2008.08.005.
Dickinson, E. (2012). Emulsion gels: The structuring of soft solids with protein-stabilized oil droplets. Food Hydrocolloids, 28 (1), 224-241. https://doi.org/10.1016/j.foodhyd.2011.12.017.
Dickinson, E., & Chen, J. (1999). Heat-set whey protein emulsion gels: Role of active and inactive filler particles. Journal of Dispersion Science and Technology, 20 (1-2), 197-213. https://doi.org/10.1080/01932699908943787.
Dille, M. J., Baydin, T., Kristiansen, K. A., & Draget, K. I. (2021a). The impact of emulsion droplet size on in vitro lipolysis rate and in vivo plasma uptake kinetics of triglycerides and vitamin D 3 in rats. Food & Function, 12 (7), 3219-3232. https://doi.org/10.1039/DOFO03386C.
Dille, M. J., & Draget, K. I. (2021). Chewable Gelatin Emulsions for Oral Lipid Delivery: Elimination of Gastric Coalescence with K-Carrageenan. European Journal of Lipid Science and Technology, 123 (2), Article 2000282. https://doi.org/10.1002/ejlt.202000282.
Dille, M. J., Draget, K. I., & Hattrem, M. N. (2015). The effect of filler particles on the texture of food gels. In Modifying Food Texture (pp. 183-200). Elsevier.
Dille, M. J., Hattrem, M. N., & Draget, K. I. (2018a). Bioactively filled gelatin gels; challenges and opportunities. Food Hydrocolloids, 76, 17-29. https://doi.org/10.1016/j.foodhyd.2016.12.028.
Dille, M. J., Hattrem, M. N., & Draget, K. I. (2018b). Soft, chewable gelatin-based pharmaceutical oral formulations: A technical approach. Pharmaceutical Development and Technology, 23 (5), 504-511. https://doi.org/10.1080/10837450.2017.1332642.
Dille, M. J., Haug, I. J., & Draget, K. I. (2021b). Gelatin and collagen. In G. Phillips, & P. Williams (Eds.), Handbook of Hydrocolloids (pp. 1073-1097). Elsevier.
Dun, H., Liang, H., Zhan, F., Wei, X., Chen, Y., Wan, J., et al., (2020). Influence of O/W emulsion on gelatinization and retrogradation properties of rice starch. Food Hydrocolloids, 103, Article 105652. https://doi.org/10.1016/j.foodhyd.2020.105652.
Farjami, T., & Madadlou, A. (2019). An overview on preparation of emulsion-filled gels and emulsion particulate gels. Trends in Food Science & Technology, 86, 85-94. https://doi.org/10.1016/j.tifs.2019.02.043.
FDA, F.a.D.A. (2018). Hazard analysis and risk-based preventive controls for human food: Draft guidance for industry. Retrieved from https://www.fda.gov/media/99572/download
Fontes-Candia, C., Ström, A., Lopez-Sanchez, P., López-Rubio, A., & Martínez-Sanz, M. (2020). Rheological and structural characterization of carrageenan emulsion gels. Algal Research, 47, Article 101873. https://doi.org/10.1016/j.algal.2020.101873.
Fraeye, I., Duvetter, T., Doungla, E., Van Loey, A., & Hendrickx, M. (2010). Fine-tuning the properties of pectin-calcium gels by control of pectin fine structure, gel composition and environmental conditions. Trends in Food Science & Technology, 21 (5), 219-228. https://doi.org/10.1016/j.tifs.2010.02.001.
Ghorbani, A., & Abedinzade, M. (2013). Comparison of in vitro and in situ methods for studying lipolysis. International Scholarly Research Notices, 2013. https://doi.org/10.1155/2013/205385.
Gravelle, A. J., & Marangoni, A. G. (2021). The influence of network architecture on the large deformation and fracture behavior of emulsion-filled gelatin gels. Food Structure, 29, Article 100193. https://doi.org/10.1016/j.foostr.2021.100193.
Hansson, A., Andersson, J., Leufven, A., & Pehrson, K. (2001). Effect of changes in pH on the release of flavour compounds from a soft drink-related model system. Food Chemistry, 74 (4), 429-435. https://doi.org/10.1016/S0308-8146 (01) 00158-3.
Hattrem, M. N., Dille, M. J., Seternes, T., Ege, T., & Draget, K. I. (2018). The relative bioavailability of ibuprofen after administration with a novel soft chewable drug formulation. Clinical Pharmacology in Drug Development, 7 (2), 168-176. https://doi.org/10.1002/cpdd.357.
Hattrem, M. N., Molnes, S., Haug, I. J., & Draget, K. I. (2015). Interfacial and rheological properties of gelatin based solid emulsions prepared with acid or alkali pretreated gelatins. Food Hydrocolloids, 43, 700-707. https://doi.org/10.1016/j.foodhyd.2014.07.026.
Haug, I. J., Draget, K. I., & Smidsrød, O. (2004). Physical and rheological properties of fish gelatin compared to mammalian gelatin. Food Hydrocolloids, 18 (2), 203-213. https://doi.org/10.1016/S0268-005X (03) 00065-1.
Haug, I. J., Sagmo, L. B., Zeiss, D., Olsen, I. C., Draget, K. I., & Seternes, T. (2011). Bioavailability of EPA and DHA delivered by gelled emulsions and soft gel capsules. European Journal of Lipid Science and Technology, 113 (2), 137-145. https://doi.org/10.1002/ejlt.201000450.
Haug, I., & Draget, K. I. (2007). W. I. P. Organization.
Hua, X., Wang, K., Yang, R., Kang, J., & Zhang, J. (2015). Rheological properties of natural low-methoxyl pectin extracted from sunflower head. Food Hydrocolloids, 44, 122-128. https://doi.org/10.1016/j.foodhyd.2014.09.026.
Imeson, A. (2009). Agar. Food Stabilisers, Thickeners and Gelling Agents, 31.
Jiang, Y., Liu, L., Wang, B., Yang, X., Chen, Z., Zhong, Y., et al., (2019). Polysaccharide-based edible emulsion gel stabilized by regenerated cellulose. Food Hydrocolloids, 91, 232-237. https://doi.org/10.1016/j.foodhyd.2019.01.028.
Karim, A. A., & Bhat, R. (2008). Gelatin alternatives for the food industry: Recent developments, challenges and prospects. Trends in Food Science & Technology, 19 (12), 644-656. https://doi.org/10.1016/j.tifs.2008.08.001.
Karuna, M., & Prasad, R. (2015). Vegetable oil-based nutraceuticals. In Plant Biology and Biotechnology (pp. 793-812). Springer.
Kim, K. H., Gohtani, S., Matsuno, R., & Yamano, Y. (1999). Effects of oil droplet and agar concentration on gel strength and microstructure of o/w emulsion gel. Journal of Texture Studies, 30 (3), 319-335. https://doi.org/10.1111/j.1745-4603.1999.tb00220.x.
Kim, K. H., Gohtani, S., & Yamano, Y. (1996). Effects of oil droplets on physical and sensory properties of o/w emulsion agar gel. Journal of Texture Studies, 27 (6), 655-670. https://doi.org/10.1111/j.1745-4603.1996.tb00999.x.
Kim, K., Gohtani, S., & Yamano, Y. (1997). Effect of oil droplets on brittleness of o/w emulsion gel. Journal of Dispersion Science and Technology, 18 (2), 199-210. https://doi.org/10.1080/01932699708943727.
Koç, H., Drake, M., Vinyard, C. J., Essick, G., van de Velde, F., & Foegeding, E. A. (2019). Emulsion filled polysaccharide gels: Filler particle effects on material properties, oral processing, and sensory texture. Food Hydrocolloids, 94, 311-325. https://doi.org/10.1016/j.foodhyd.2019.03.018.
Lahrech, K., Safouane, A., & Peyrellasse, J. (2005). Sol state formation and melting of agar gels rheological study. Physica A: Statistical Mechanics and its Applications, 358 (1), 205-211. https://doi.org/10.1016/j.physa.2005.06.022.
Lamothe, S., Desroches, V., & Britten, M. (2019). Effect of milk proteins and food-grade surfactants on oxidation of linseed oil-in-water emulsions during in vitro digestion. Food Chemistry, 294, 130-137. https://doi.org/10.1016/j.foodchem.2019.04.107.
Lamothe, S., Jolibois, É., & Britten, M. (2020). Effect of emulsifiers on linseed oil emulsion structure, lipolysis and oxidation during in vitro digestion. Food & Function, 11 (11), 10126-10136. https://doi.org/10.1039/d0fo02072a.
Larsen, A. T., Sassene, P., & Müllertz, A. (2011). In vitro lipolysis models as a tool for the characterization of oral lipid and surfactant based drug delivery systems. International Journal of Pharmaceutics, 417 (1-2), 245-255. https://doi.org/10.1016/j.ijpharm.2011.03.002.
Leitzmann, C. (2014). Vegetarian nutrition: Past, present, future. The American Journal of Clinical Nutrition, 100 (suppl_1), 496S-502S. https://doi.org/10.3945/ajcn.113.071365.
Lestari, W., Octavianti, F., Jaswir, I., & Hendri, R. (2019). Plant-based substitutes for gelatin. Contemporary Management and Science Issues in the Halal Industry, 319-322. https://doi.org/10.1007/978-981-13-2677-6_26.
Levi ć, S., Lijakovi ć, I. P., Ðor [symbol] evicć, V., Rac, V., Raki ć, V., Knudsen, T. Š., et al., (2015). Characterization of sodium alginate/D-limonene emulsions and respective calcium alginate/D-limonene beads produced by electrostatic extrusion. Food Hydrocolloids, 45, 111-123. https://doi.org/10.1016/j.foodhyd.2014.10.001.
Li, Y., Hu, M., & McClements, D. J. (2011). Factors affecting lipase digestibility of emulsified lipids using an in vitro digestion model: Proposal for a standardised pHstat method. Food Chemistry, 126 (2), 498-505. https://doi.org/10.1016/j.foodchem.2010.11.027.
Lin, D., Kelly, A. L., Maidannyk, V., & Miao, S. (2020a). Effect of concentrations of alginate, soy protein isolate and sunflower oil on water loss, shrinkage, elastic and structural properties of alginate-based emulsion gel beads during gelation. Food Hydrocolloids, 108, Article 105998. https://doi.org/10.1016/j.foodhyd.2020.105998.
Lin, D., Kelly, A. L., & Miao, S. (2020b). Preparation, structure-property relationships and applications of different emulsion gels: Bulk emulsion gels, emulsion gel particles, and fluid emulsion gels. Trends in Food Science & Technology, 102, 123-137. https://doi.org/10.1016/j.tifs.2020.05.024.
Lin, X., Wang, Q., Li, W., & Wright, A. J. (2014). Emulsification of algal oil with soy lecithin improved DHA bioaccessibility but did not change overall in vitro digestibility. Food & Function, 5 (11), 2913-2921. https://doi.org/10.1039/C4FO00577E.
Lin, X., & Wright, A. J. (2018). Pectin and gastric pH interactively affect DHA-rich emulsion in vitro digestion microstructure, digestibility and bioaccessibility. Food Hydrocolloids, 76, 49-59. https://doi.org/10.1016/j.foodhyd.2017.06.010.
Lu, Y., Mao, L., Hou, Z., Miao, S., & Gao, Y. (2019). Development of emulsion gels for the delivery of functional food ingredients: From structure to functionality. Food Engineering Reviews, 11 (4), 245-258. https://doi.org/10.1007/s12393-019-09194-z.
Lupi, F. R., Gabriele, D., Seta, L., Baldino, N., de Cindio, B., & Marino, R. (2015). Rheological investigation of pectin-based emulsion gels for pharmaceutical and cosmetic uses. Rheologica Acta, 54 (1), 41-52. https://doi.org/10.1007/s00397-014-0809-8.
Mazumder, P., Roopa, B., & Bhattacharya, S. (2007). Textural attributes of a model snack food at different moisture contents. Journal of Food Engineering, 79 (2), 511-516. https://doi.org/10.1016/j.jfoodeng.2006.02.011.
McClements, D. J. (2018). Enhanced delivery of lipophilic bioactives using emulsions: A review of major factors affecting vitamin, nutraceutical, and lipid bioaccessibility. Food & Function, 9 (1), 22-41. https://doi.org/10.1039/C7FO01515A.
Mella, C., Quilaqueo, M., Zúñiga, R. N., & Troncoso, E. (2021). Impact of the simulated gastric digestion methodology on the In vitro intestinal proteolysis and lipolysis of emulsion gels. Foods (Basel, Switzerland), 10 (2), 321. https://doi.org/10.3390/foods10020321.
Minekus, M., Alminger, M., Alvito, P., Ballance, S., Bohn, T., Bourlieu, C., et al., (2014). A standardised static in vitro digestion method suitable for food—an international consensus. Food & Function, 5 (6), 1113-1124. https://doi.org/10.1039/C3FO60702J.
Mizrahi, S. (2010). Syneresis in food gels and its implications for food quality. In Chemical Deterioration and Physical Instability of Food and Beverages (pp. 324-348). Elsevier.
Ngouémazong, E. D., Christiaens, S., Shpigelman, A., Van Loey, A., & Hendrickx, M. (2015). The emulsifying and emulsion-stabilizing properties of pectin: A review. Comprehensive Reviews in Food Science and Food Safety, 14 (6), 705-718. https://doi.org/10.1111/1541-4337.12160.
Noguerol, A. T., Pagán, M. J., García-Segovia, P., & Varela, P. (2021). Green or clean? Perception of clean label plant-based products by omnivorous, vegan, vegetarian and flexitarian consumers. Food Research International, 149, Article 110652. https://doi.org/10.1016/j.foodres.2021.110652.
Pons, M., & Fiszman, S. (1996). Instrumental texture profile analysis with particular reference to gelled systems. Journal of Texture Studies, 27 (6), 597-624. https://doi.org/10.1111/j.1745-4603.1996.tb00996.x.
Rao, M., Van Buren, J., & Cooley, H. (1993). Rheological changes during gelation of high-methoxyl pectin/fructose dispersions: Effect of temperature and aging. Journal of Food Science, 58 (1), 173-176. https://doi.org/10.1111/j.1365-2621.1993.tb03237.x.
Read, N., Al-Janabi, M., Holgate, A., Barber, D., & Edwards, C. (1986). Simultaneous measurement of gastric emptying, small bowel residence and colonic filling of a solid meal by the use of the gamma camera. Gut, 27 (3), 300-308. https://doi.org/10.1136/gut.27.3.300.
Rosenthal, A. J. (2010). Texture profile analysis-how important are the parameters? Journal of Texture Studies, 41 (5), 672-684. https://doi.org/10.1111/j.1745-4603.2010.00248.x.
Saha, D., & Bhattacharya, S. (2010). Characteristics of gellan gum based food gels. Journal of Texture Studies, 41 (4), 459-471. https://doi.org/10.1111/j.1745-4603.2010.00236.x.
Sala, G., de Wijk, R. A., van de Velde, F., & van Aken, G. A. (2008). Matrix properties affect the sensory perception of emulsion-filled gels. Food Hydrocolloids, 22 (3), 353-363. https://doi.org/10.1016/j.foodhyd.2006.12.009.
Sala, G., van de Velde, F., Stuart, M. A. C., & van Aken, G. A. (2007). Oil droplet release from emulsion-filled gels in relation to sensory perception. Food Hydrocolloids, 21 (5-6), 977-985. https://doi.org/10.1016/j.foodhyd.2006.08.009.
Sala, G., Van Vliet, T., Stuart, M. A. C., Van Aken, G. A., & Van de Velde, F. (2009). Deformation and fracture of emulsion-filled gels: Effect of oil content and deformation speed. Food Hydrocolloids, 23 (5), 1381-1393. https://doi.org/10.1016/j.foodhyd.2008.11.016.
Santini, A., & Novellino, E. (2017). To nutraceuticals and back: Rethinking a concept. Foods (Basel, Switzerland), 6 (9), 74. https://doi.org/10.3390/foods6090074.
Sarkar, A., Juan, J.-M., Kolodziejczyk, E., Acquistapace, S., Donato-Capel, L., & Wooster, T. J. (2015). Impact of protein gel porosity on the digestion of lipid emulsions. Journal of Agricultural and Food Chemistry, 63 (40), 8829-8837. https://doi.org/10.1021/acs.jafc.5b03700.
Schrieber, R., & Gareis, H. (2007). Gelatine handbook: Theory and industrial practice. John Wiley & Sons.
Šeremet, D., Mandura, A., Cebin, A. V., Martini ć, A., Gali ć, K., & Komes, D. (2020). Challenges in confectionery industry: Development and storage stability of innovative white tea-based candies. Journal of Food Science, 85 (7), 2060-2068. https://doi.org/10.1111/1750-3841.15306.
Shao, P., Feng, J., Sun, P., Xiang, N., Lu, B., & Qiu, D. (2020). Recent advances in improving stability of food emulsion by plant polysaccharides. Food Research International, 137, Article 109376. https://doi.org/10.1016/j.foodres.2020.109376.
Sørensen, A.-D. M., Haahr, A.-M., Becker, E. M., Skibsted, L. H., Bergenståhl, B., Nilsson, L., et al., (2008). Interactions between iron, phenolic compounds, emulsifiers, and pH in omega-3-enriched oil-in-water emulsions. Journal of Agricultural and Food Chemistry, 56 (5), 1740-1750. https://doi.org/10.1021/jf072946z.
Sousa, A. M., Rocha, C. M., & Gonçalves, M. P. (2021). Agar. In Handbook of Hydrocolloids (pp. 731-765). Elsevier.
Stannard, S. (2018). Consumer Insights. Retrieved from https://media.ahdb.org.uk/media/Default/Consumer % 20 and %20Retail %20Insight %20Image s/PDF % 20articles/ConsumerInsights %20WEB_1653_180725.pdf
Van den Bosch, E., & Gielens, C. (2003). Gelatin degradation at elevated temperature. International Journal of Biological Macromolecules, 32 (3-5), 129-138. https://doi.org/10.1016/S0141-8130 (03) 00046-1.
Van Vliet, T., & Walstra, P. (2017). Dispersed Systems Basic Considerations. In Fennema's Food Chemistry (pp. 467-539). CRC Press.
Wang, Z., Neves, M. A., Kobayashi, I., Uemura, K., & Nakajima, M. (2013). Preparation, characterization, and in vitro gastrointestinal digestibility of oil-in-water emulsion-agar gels. Bioscience, Biotechnology, and Biochemistry, Article 120659. https://doi.org/10.1271/bbb.120659.
Weiss, J., Scherze, I., & Muschiolik, G. (2005). Polysaccharide gel with multiple emulsion. Food Hydrocolloids, 19 (3), 605-615. https://doi.org/10.1016/j.foohyd.2004.10.023.
Worsøe, J., Fynne, L., Gregersen, T., Schlageter, V., Christensen, L. A., & Dahlerup, J. F. (2011). Gastric transit and small intestinal transit time and motility assessed by a magnet tracking system. BMC Gastroenterology, 11 (1), 1-10. https://doi.org/10.1186/1471-230X-11-145.
Yamano, Y., Kagawa, Y., Kim, K.-H., & Gohtani, S. (1996). Stability and uniformity of oil droplets in preparation of O/W emulsion agar gel. Food Science and Technology International, Tokyo, 2 (1), 16-18. https://doi.org/10.3136/fsti9596t9798.2.16.
Yang, J., Wan, L., Duan, X., Wang, H., Yang, Z., Liu, F., et al., (2022). Potential low-calorie model that inhibits free fatty acid release and helps curcumin deliver in vitro: Ca²⁺-induced emulsion gels from low methyl-esterified pectin with the presence of erythritol. International Journal of Biological Macromolecules. https://doi.org/10.1016/j.ijbiomac.2022.01.069.
Yang, X., Gong, T., Li, D., Li, A., Sun, L., & Guo, Y. (2019). Preparation of high viscoelastic emulsion gels based on the synergistic gelation mechanism of xanthan and konjac glucomannan. Carbohydrate Polymers, 226, Article 115278. https://doi.org/10.1016/j.carbpol.2019.115278.
Yue, J., Chen, X., Yao, X., Gou, Q., Li, D., Liu, H., et al., (2022). Stability improvement of emulsion gel fabricated by Artemisia sphaerocephala Krasch. Polysaccharide fractions. International Journal of Biological Macromolecules, 205, 253-260. https://doi.org/10.1016/j.ijbiomac.2022.02.069.
Zeeb, B., Roth, M., & Endreß, H.-U. (2021). Commercial pectins. In Handbook of Hydrocolloids (pp. 295-315). Elsevier.
Zhang, L., Zheng, J., Wang, Y., Ye, X., Chen, S., Pan, H., et al., (2022). Fabrication of rhamnogalacturonan-I enriched pectin-based emulsion gels for protection and sustained release of curcumin. Food Hydrocolloids, 128, Article 107592. https://doi.org/10.1016/j.foodhyd.2022.107592.

### Exemplary Aspects

In view of the described compositions, devices, systems, and methods, herein below are described certain more particularly described aspects of the inventions. The particularly recited aspects should not, however, be interpreted to have any limiting effect on any different claims containing different or more general teachings described herein or that the “particular” aspects are somehow limited in some way other than the inherent meanings of the language and formulas literally used therein.

Example 1. An orally administrable, gelled oil-in-water emulsion in unit dose form, wherein the gelled oil-in-water emulsion is a self-supporting, viscoelastic solid having a water activity in the range of about 0.4 to 0.9 and which comprises a gelled aqueous phase having a pH of 3 to 5.5, and wherein the gelled aqueous phase comprises a gelling agent which is a low-methoxy amidated pectin and the gelled oil-in-water emulsion is stabilised by an emulsifier which is a phospholipid or mixture of phospholipids.

Example 2. An orally administrable, gelled oil-in-water emulsion of any examples herein, particularly example 1, wherein the low-methoxy amidated pectin has a degree of esterification of less than 40%.

Example 3. An orally administrable, gelled oil-in-water emulsion of any examples herein, particularly example 1, wherein the low-methoxy amidated pectin has a degree of esterification in the range of from 20% to 30%.

Example 4. An orally administrable, gelled oil-in-water emulsion of any examples herein, particularly examples 1 to 3, wherein the low-methoxy amidated pectin has a degree of amidation in the range of from 15% to 25%.

Example 5. An orally administrable, gelled oil-in-water emulsion of any examples herein, particularly examples 1 to 3, wherein the low-methoxy amidated pectin has a degree of amidation in the range of 20% to 25%.

Example 6. An orally administrable, gelled oil-in-water emulsion of any examples herein, particularly example 1, wherein the low-methoxy amidated pectin has a degree of esterification in the range of from 20% to 30% and a degree of amidation in the range of from 20% to 25%.

Example 7. An orally administrable, gelled oil-in-water emulsion of any examples herein, particularly any one of examples 1 to 6, which further comprises calcium ions.

Example 8. An orally administrable, gelled oil-in-water emulsion of any examples herein, particularly example 7, wherein the calcium ions are present in the aqueous phase at a concentration in the range of up to 250 mM, preferably 10 to 200 mM, e.g. 25 to 150 mM.

Example 9. An orally administrable, gelled oil-in-water emulsion of any examples herein, particularly examples 1 to 6, which is substantially free from any calcium ions.

Example 10. An orally administrable, gelled oil-in-water emulsion of any examples herein, particularly examples 1 to 9, wherein the emulsifier is lecithin or a lecithin derivative.

Example 11. An orally administrable, gelled oil-in-water emulsion of any examples herein, particularly example 10, wherein said lecithin is sunflower lecithin or a derivative thereof.

Example 12. An orally administrable, gelled oil-in-water emulsion of any examples herein, particularly example 10 or example 11, wherein the lecithin derivative is an enzymatically hydrolysed lecithin.

Example 13. An orally administrable, gelled oil-in-water emulsion of any examples herein, particularly examples 1 to 12, wherein the emulsifier is present at a concentration in the range from 0.01 to 0.5 wt. %, preferably from 0.05 to 0.4 wt. %, particularly from 0.1 to 0.3 wt. %, e.g. from 0.2 to 0.3 wt. % (based on the total weight of the overall composition).

Example 14. An orally administrable, gelled oil-in-water emulsion of any examples herein, particularly examples 1 to 13, wherein the gelled aqueous phase of the emulsion has a pH in the range from 3.5 to 5, preferably 3.75 to 4.75.

Example 15. An orally administrable, gelled oil-in-water emulsion of any examples herein, particularly examples 1 to 14, wherein said aqueous phase constitutes 60 to 99 wt. %, preferably from 70 to 95 wt. %, for example from 75 to 90 wt. %, from 75 to 85 wt. %, or from 75 to 80 wt. % of the composition.

Example 16. An orally administrable, gelled oil-in-water emulsion of any examples herein, particularly examples 1 to 15, wherein the aqueous phase further comprises one or more bulking agents, for example sugar alcohols or sugars.

Example 17. An orally administrable, gelled oil-in-water emulsion of any examples herein, particularly example 16, wherein said bulking agents are present at a concentration of from 45 to 70 wt. %, preferably 50 to 65 wt. %, e.g. 55 to 60 wt. %, based on the aqueous phase.

Example 18. An orally administrable, gelled oil-in-water emulsion of any examples herein, particularly examples 1 to 17, having an oil phase which comprises one or more physiologically tolerable lipids derived from rapeseed oil, sunflower oil, corn oil, olive oil, sesame oil, palm kernel oil, coconut oil, a nut oil, algae oil or hemp oil.

Example 19. An orally administrable, gelled oil-in-water emulsion of any examples herein, particularly examples 1 to 18, having an oil phase which constitutes from 1 to 40 wt. %, preferably from 5 to 30 wt. %, for example from 10 to 25 wt. %, from 15 to 25 wt. % or from 20 to 25 wt. % of the gelled oil-in-water emulsion.

Example 20. An orally administrable, gelled oil-in-water emulsion of any examples herein, particularly examples 1 to 19, which further comprises at least one pharmaceutically active agent and/or at least one nutraceutically active agent.

Example 21. An orally administrable, gelled oil-in-water emulsion of any examples herein, particularly examples 1 to 20, wherein said unit dose form is uncoated.

Example 22. A package comprising an air-tight and light-tight compartment containing one dose unit of the gelled oil-in-water emulsion of any examples herein, particularly examples 1 to 21.

Example 23. A method for the preparation of an orally administrable, gelled oil-in-water emulsion of any examples herein, particularly examples 1 to 21, said method comprising the steps of: forming an oil phase which comprises one or more physiologically tolerable lipids; forming an aqueous phase comprising a gelling agent which is a low-methoxy amidated pectin; combining said oil phase and said aqueous phase to form an oil-in-water emulsion in the presence of an emulsifier which is phospholipid or mixture of phospholipids; and allowing said emulsion to gel.

Example 24. A gelled oil-in-water emulsion of any examples herein, particularly examples 1 to 21, for oral use as a medicament or for oral use in therapy.

Example 25. Use of a gelled oil-in-water emulsion of any examples herein, particularly examples 1 to 21, as a nutraceutical.

Example 26. A method of treatment of a human or non-human animal subject (e.g. a patient) to combat a condition responsive to a pharmaceutically active agent, said method comprising the step of orally administering to said subject a pharmaceutically effective amount of said agent in the form of a gelled oil-in-water emulsion of any examples herein, particularly examples 1 to 21.

1. An orally administrable, gelled oil-in-water emulsion in unit dose form, wherein the gelled oil-in-water emulsion is a self-supporting, viscoelastic solid having a water activity in the range of about 0.4 to 0.9 and which comprises a gelled aqueous phase having a pH of 3 to 5.5, and wherein the gelled aqueous phase comprises a gelling agent which is a low-methoxy amidated pectin and the gelled oil-in-water emulsion is stabilised by an emulsifier which is a phospholipid or mixture of phospholipids.

2. The orally administrable, gelled oil-in-water emulsion as claimed in claim 1, wherein the low-methoxy amidated pectin has a degree of esterification of less than 40%.

3. The orally administrable, gelled oil-in-water emulsion as claimed in claim 1, wherein the low-methoxy amidated pectin has a degree of esterification in the range of from 20% to 30%.

4. The orally administrable, gelled oil-in-water emulsion as claimed in claim 1, wherein the low-methoxy amidated pectin has a degree of amidation in the range of from 15% to 25%.

5. The orally administrable, gelled oil-in-water emulsion as claimed in claim 1, wherein the low-methoxy amidated pectin has a degree of amidation in the range of 20% to 25%.

6. The orally administrable, gelled oil-in-water emulsion as claimed in claim 1, wherein the low-methoxy amidated pectin has a degree of esterification in the range of from 20% to 30% and a degree of amidation in the range of from 20% to 25%.

7. The orally administrable, gelled oil-in-water emulsion as claimed in claim 1, which further comprises calcium ions, wherein the calcium ions are present in the aqueous phase at a concentration in the range of up to 250 mM.

8. The orally administrable, gelled oil-in-water emulsion as claimed in claim 1, which is substantially free from any calcium ions.

9. The orally administrable, gelled oil-in-water emulsion as claimed in claim 1, wherein the emulsifier is a lecithin or a lecithin derivative.

10. The orally administrable, gelled oil-in-water emulsion as claimed in claim 1, wherein the emulsifier is present at a concentration of from 0.01 to 0.5 wt. % (based on the total weight of the overall composition).

11. The orally administrable, gelled oil-in-water emulsion as claimed in claim 1, wherein the gelled aqueous phase of the emulsion has a pH in the range from 3.5 to 5.

12. The orally administrable, gelled oil-in-water emulsion as claimed in claim 1, wherein said aqueous phase constitutes 60 to 99 wt. % of the composition.

13. The orally administrable, gelled oil-in-water emulsion as claimed in claim 1, wherein the aqueous phase further comprises one or more bulking agents selected from sugar alcohols and sugars.

14. The orally administrable, gelled oil-in-water emulsion as claimed in claim 13, wherein said bulking agents are present at a concentration of from 45 to 70 wt. %, based on the aqueous phase.

15. The orally administrable, gelled oil-in-water emulsion as claimed in claim 1, having an oil phase which comprises one or more physiologically tolerable lipids derived from rapeseed oil, sunflower oil, corn oil, olive oil, sesame oil, palm kernel oil, coconut oil, a nut oil, algae oil, or hemp oil.

16. The orally administrable, gelled oil-in-water emulsion as claimed in claim 1, having an oil phase which constitutes from 1 to 40 wt. % of the gelled oil-in-water emulsion.

17. The orally administrable, gelled oil-in-water emulsion as claimed in claim 1, having an oil phase which constitutes from 5 to 30 wt. % of the gelled oil-in-water emulsion.

18. The orally administrable, gelled oil-in-water emulsion as claimed in claim 1, which further comprises at least one pharmaceutically active agent and/or at least one nutraceutically active agent.

19. The orally administrable, gelled oil-in-water emulsion as claimed in claim 1, wherein said unit dose form is uncoated.

20. A package comprising an air-tight and light-tight compartment containing one dose unit of the gelled oil-in-water emulsion as claimed in claim 1.
